(19) United States
(12) Patent Application Publication
(10) Pub. No.: US 2005/0158324 A1
(43) Pub. Date: Jul. 21, 2005

(54) Methods of therapy and diagnosis using targeting of cells that express BCLP polypeptides

(76) Inventor: Peter C.R. Emtage, Sunnyvale, CA (US)

Correspondence Address: NUVELO, INC, 675 ALMANOR AVE., SUNNYVALE, CA, 94085, US

(21) Appl. No.: 11/014,487

(22) Filed: Dec. 15, 2004

Related U.S. Application Data
(63) Continuation-in-part of application No. 10/737,666, filed on Dec. 15, 2003.

(51) Int. Cl. C12Q 1/68; G01N 33/574; A61K 39/395

(52) U.S. Cl. 424/155.1; 424/178.1; 435/6; 435/7.23

(57) ABSTRACT

Certain cells, including cancer cells such as cells from cancers of the colon, breast, lung, ovary, prostate, pancreas and skin are capable of expressing BCLP. Targeting using BCLP polypeptides, nucleic acids encoding for BCLP polypeptides, anti-BCLP antibodies, peptides and small molecules provides a method of killing or inhibiting the growth of the cancer cells that express the BCLP protein. Methods for the diagnosis and therapy of tumors that express BCLP are described.

### BACKGROUND

### 1.1 CROSS REFERENCE TO RELATED APPLICATIONS

This application claims the benefit of priority to and is a continuation-in-part application of U.S. patent application Ser. No. 10/737,666 filed Dec. 15, 2003 entitled “Methods of Therapy and Diagnosis Using Targeting of Cells that Express BCLP Polypeptides” Attorney Docket No. NUVO-11, herein incorporated by reference in its entirety.

### 1.2 TECHNICAL FIELD

This invention relates to compositions and methods for targeting BCLP-expressing cells using antibodies, polypeptides, polynucleotides, peptides, and small molecules and their use in the therapy and diagnosis of various pathological states, including cancers such as colon, breast, lung, ovarian, prostate, pancreatic cancers, and melanoma.

### 1.3 SEQUENCE LISTING

The sequences of the polynucleotide and polypeptide of the invention are listed in the sequence listing and are submitted on a compact disc containing the file labeled “NUVO-11CP.txt”—52.0 KB (53,248 bytes), which was created on an IBM PC, Windows 2000 operating system on Monday Dec. 13, 2004 at 10:18 AM. The sequence listing entitled “NUVO-11 CP.txt” is herein incorporated by reference in its entirety. A computer readable format (“CRF”) and three duplicate copies (“Copy 1,” “Copy 2” and “Copy 3”) of the Sequence Listing “NUVO-11CP.txt” are submitted herein. Applicants hereby state that the content of the CRF and Copies 1, 2 and 3 of the Sequence Listing, submitted in accordance with 37 CFR §1.821(c) and (e), respectively, are the same.

### 1.4 BACKGROUND ART

Antibody therapy for cancer involves the use of antibodies, or antibody fragments, against a tumor antigen to target antigen-expressing cells. Antibodies, or antibody fragments, may have direct or indirect cytotoxic effects or may be conjugated or fused to cytotoxic moieties. Direct effects include the induction of apoptosis, the blocking of growth factor receptors, and anti-idiotype antibody formation. Indirect effects include antibody-dependent cell-mediated cytotoxicity (ADCC) and complement-mediated cellular cytotoxicity (CMCC). When conjugated or fused to cytotoxic moieties, the antibodies, or fragments thereof, provide a method of targeting the cytotoxicity towards the tumor antigen expressing cells. (Green, et al., Cancer Treatment Reviews, 26:269-286 (2000), incorporated herein by reference in its entirety).

Because antibody therapy targets cells expressing a particular antigen, there is a possibility of cross-reactivity with normal cells or tissue. Although some cells, such as hematopoietic cells, are readily replaced by precursors, cross-reactivity with many tissues can lead to detrimental results. Thus, considerable research has gone towards finding tumor-specific antigens. Such antigens are found almost exclusively on tumors or are expressed at a greater level in tumor cells than the corresponding normal tissue. Tumor-specific antigens provide targets for antibody targeting of cancer, or other disease-related cells, expressing the antigen. Antibodies specific to such tumor-specific antigens can be conjugated to cytotoxic compounds or can be used alone in immunotherapy. Immunotoxins target cytotoxic compounds to induce cell death. For example, anti-CD22 antibodies conjugated to deglycosylated ricin A may be used for treatment of B cell lymphoma that has relapsed after conventional therapy (Amlot, et al., Blood 82:2624-2633 (1993), incorporated herein by reference in its entirety) and has demonstrated encouraging responses in initial clinical studies.

The immune system functions to eliminate organisms or cells that are recognized as non-self, including microorganisms, neoplasms and transplants. A cell-mediated host response to tumors includes the concept of immunologic surveillance, by which cellular mechanisms associated with cell-mediated immunity, destroy newly transformed tumor cells after recognizing tumor-associated antigens (antigens associated with tumor cells that are not apparent on normal cells). Furthermore, a humoral response to tumor-associated antigens enables destruction of tumor cells through immunological processes triggered by the binding of an antibody to the surface of a cell, such as antibody-dependent cellular cytotoxicity (ADCC) and complement mediated lysis.

Recognition of an antigen by the immune system triggers a cascade of events including cytokine production, B-cell proliferation, and subsequent antibody production. Often tumor cells have reduced capability of presenting antigen to effector cells, thus impeding the immune response against a tumor-specific antigen. In some instances, the tumor-specific antigen may not be recognized as non-self by the immune system, preventing an immune response against the tumor-specific antigen from occurring. In such instances, stimulation or manipulation of the immune system provides effective techniques of treating cancers expressing one or more tumor-specific antigens.

For example, Rituximab (Rituxan®) is a chimeric antibody directed against CD20, a B cell-specific surface molecule found on >95% of B-cell non-Hodgkin's lymphoma (Press, et al., Blood 69:584-591 (1987); Malony, et al., Blood 90:2188-2195 (1997), both of which are incorporated herein in their entirety). Rituximab induces ADCC and inhibits cell proliferation through apoptosis in malignant B cells in vitro (Maloney, et al., Blood 88:637a (1996), incorporated herein by reference in its entirety). Rituximab is currently used as a therapy for advanced stage or relapsed low-grade non-Hodgkin's lymphoma, which has not responded to conventional therapy.

Active immunotherapy, whereby the host is induced to initiate an immune response against its own tumor cells can be achieved using therapeutic vaccines. One type of tumor-specific vaccine uses purified idiotype protein isolated from tumor cells, coupled to keyhole limpet hemocyanin (KLH) and mixed with adjuvant for injection into patients with low-grade follicular lymphoma (Hsu, et al., Blood 89:3129-3135 (1997), incorporated herein by reference in its entirety). Another type of vaccine uses antigen-presenting cells (APCs), which present antigen to naïve T cells during the recognition and effector phases of the immune response. Dendritic cells, one type of APC, can be used in a cellular vaccine in which the dendritic cells are isolated from the patient, co-cultured with tumor antigen and then reinfused as a cellular vaccine (Hsu, et al., Nat. Med. 2:52-58 (1996), incorporated herein by reference in its entirety). Immune responses can also be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002)).

Cancer of the colon, breast, lung, ovary, pancreas, prostate and skin as well as other cancers are treatable and often curable diseases when localized to the respective organs. Surgery is the primary form of treatment and results in cure in many patients. However, recurrence following surgery is a major problem and often is the ultimate cause of death. Systemic adjuvant chemotherapy reduces the recurrence rate and prolongs the survival of patients that present with late stage disease. However, the toxic effects of therapeutic outcomes, and the presence of drug refractoriness remain considerable problems that need to be overcome to improve the quality of life and reduce the death rate of cancer patients. A number of approaches using vaccines and antibodies as adjuvant therapy are being studied, and mounting evidence indicates that many cancers are immunogenic, and that they may reasonably be considered as a targets for immunotherapy. Antibody-based therapy has been effective in the treatment of certain cancers. For example, HERCEPTIN® (Genetech, Calif.) antibodies have been used successfully to treat some cancers of the breast, and edrecolomab (Panorex®) has been shown to be a less toxic and adequate alternative to chemotherapy for patients with stage II colon cancer (Yves Dencausse^{et} al., Annals of Oncology, Vol 11, Suppl.4 October 2000, page 47).

In spite of these advances, the deployment of immunotherapy as a treatment option against cancers remains hampered by the lack of tumor associated antigens that are tumor-specific, strongly immunogenic and that are shared among different patients (Dalerba et al., Clin Rev Oncol Hematol 46:33-57 (2003)). Therefore, there exists a need in the art to identify antigens that are clearly and specifically expressed on the surface of cancer cells that could serve as targets for various targeting strategies. Accordingly, Applicants have identified a molecular target useful for therapeutic intervention in colon, breast, lung, ovary, pancreas, prostate and skin cancer, and provide herein methods for the diagnosis and therapy of said cancers.

### 2. SUMMARY OF THE INVENTION

The invention provides compositions and therapeutic and diagnostic methods of targeting cells expressing Beta Casein Like Protein (BCLP) by using targeting elements such as BCLP polypeptides, nucleic acids encoding a BCLP protein, and anti-BCLP antibodies, including fragments or other modifications thereof, peptides and small molecules. BCLP herein refers to a BCLP protein that comprises the polypeptide of SEQ ID NO: 18, which is encoded by the polynucleotide of SEQ ID NO: 19.

BCLP is expressed at very high levels in tumors of the colon, breast, lung, ovary, pancreas, prostate, and melanoma cells relative to its expression in healthy organs including colon, lung, kidney, small intestine, brain, pancreas, and adrenal gland. Thus, targeting of cells that express BCLP will destroy or inhibit the growth of BCLP-expressing cancer cells while having a minimal or no effect on other healthy cells and tissues. Disorders in which other cells express BCLP may benefit from BCLP targeting therapy. For example inhibition of growth and/or destruction of BCLP-expressing cancer cells results from targeting such cells with anti-BCLP antibodies. One embodiment of the invention is a method of destroying BCLP-expressing cells by conjugating anti-BCLP antibodies with cytocidal materials such as radioisotopes or other cytotoxic compounds

The present invention provides a variety of targeting elements and compositions. One such embodiment is a composition comprising an anti-BCLP antibody preparation. Exemplary antibodies include a single anti-BCLP antibody, a combination of two or more anti-BCLP antibodies, a combination of an anti-BCLP antibody with a non-BCLP antibody, a combination of anti-BCLP antibody and a therapeutic agent, an anti-BCLP-antibody linked to a prodrug-activating enzyme, a combination of an anti-BCLP antibody and a cytocidal agent, a bispecific anti-BCLP antibody, Fab BCLP antibodies or fragments thereof, including any fragment of an antibody that retains one or more complementarity determining regions (CDRs) that recognize BCLP, humanized anti-BCLP antibodies that retain all or a portion of a CDR that recognizes BCLP, anti-BCLP conjugates, and anti-BCLP antibody fusion proteins.

Another targeting embodiment of the invention is a composition comprising a BCLP antigen, or a fragment or variant thereof, and optionally comprising a suitable adjuvant.

Another targeting embodiment is a preparation comprising a BCLP polypeptide, or peptide fragment thereof. A further targeting embodiment is a non-BCLP polypeptide or peptide that binds BCLP.

Another targeting embodiment is a preparation comprising a small molecule that recognizes BCLP.

Yet another targeting embodiment is a preparation comprising a nucleic acid encoding BCLP, or a fragment or variant thereof, optionally within a recombinant vector. A further targeting embodiment of the present invention is a composition comprising an antigen-presenting cell transformed with a nucleic acid encoding BCLP, or a fragment or variant thereof, optionally within a recombinant vector.

The invention also provides a method of killing or inhibiting the growth of cancer cells, including colon, breast, lung, ovary, pancreas, prostate, and melanoma cancer cells, BCLP-expressing cancer cells, which comprises administering a targeting element or a targeting composition in an amount effective to inhibit the growth of said cancer cells. Any one of the targeting elements or compositions described herein may be used in such methods, including an anti-BCLP antibody preparation, a vaccine or composition comprising a BCLP polypeptide, fragment, or variant thereof, composition of a nucleic acid encoding BCLP, or fragment or variant thereof, optionally within a recombinant vector, or a composition of an antigen-presenting cell transformed with a nucleic acid encoding BCLP, or fragment or variant thereof, optionally within a recombinant vector, or a BCLP polypeptide, peptide fragment, or variant thereof, or a binding polypeptide, peptide or small molecule that binds to BCLP. Similarly, non-solid type tumors such as hematopoietic-based tumors can be targeted if they bear the BCLP antigen.

The present invention further provides a method of treating disorders associated with the proliferation of BCLP-expressing cells in a subject in need thereof, comprising the step of administering a targeting element or targeting composition in a therapeutically effective amount to treat disorders associated with BCLP-expressing cells.

Any one of the targeting elements or compositions described herein may be used in such methods, including an anti-BCLP antibody preparation, a vaccine or composition comprising a BCLP polypeptide, or a fragment or variant thereof or a composition of a nucleic acid encoding BCLP, or a fragment or variant thereof, optionally with a recombinant vector or a composition of an antigen-presenting cell transformed with a nucleic acid encoding BCLP, or fragment or variant thereof, optionally within a recombinant vector, or a BCLP polypeptide, peptide fragment or variant thereof, or a binding polypeptide, peptide or small molecule that binds to a BCLP of the invention.

The invention further provides a method of modulating the immune system by either suppression or stimulation of growth factors and cytokines, by administering the targeting elements or compositions of the invention. The invention also provides a method of modulating the immune system through activation of immune cells (such as natural killer cells, T cells, B cells and myeloid cells), through the suppression of activation, or by stimulating or suppressing proliferation of these cells by BCLP peptide fragments or BCLP antibodies.

The present invention thereby provides a method of treating immune-related disorders by suppressing the immune system in a subject in need thereof, by administering the targeting elements or compositions of the invention. Such immune-related disorders include but are not limited to autoimmune disease and organ transplant rejection.

The present invention also provides a method of diagnosing disorders associated with BCLP-expressing cells comprising the step of measuring the expression patterns of BCLP protein and/or its associated mRNA. Yet another embodiment of a method of diagnosing disorders associated with BCLP-expressing cells comprising the step of detecting BCLP expression using anti-BCLP antibodies. Expression levels or patterns may then be compared with a suitable standard indicative of the desired diagnosis. Such methods of diagnosis include compositions, kits and other approaches for determining whether a patient is a candidate for BCLP targeting therapy in which said BCLP is targeted.

The present invention also provides a method of enhancing the effects of therapeutic agents and adjunctive agents used to treat and manage disorders associated with BCLP-expressing cells, by administering BCLP preparations of said BCLP with therapeutic and adjuvant agents commonly used to treat such disorders.

### 3. BRIEF DESCRIPTION OF THE DRAWINGS

FIG. 1 depicts the nucleic acid sequence of a cDNA (SEQ ID NO: 1; accession no. gi:34147466) encoding the BCLP polypeptide of SEQ ID NO: 2.

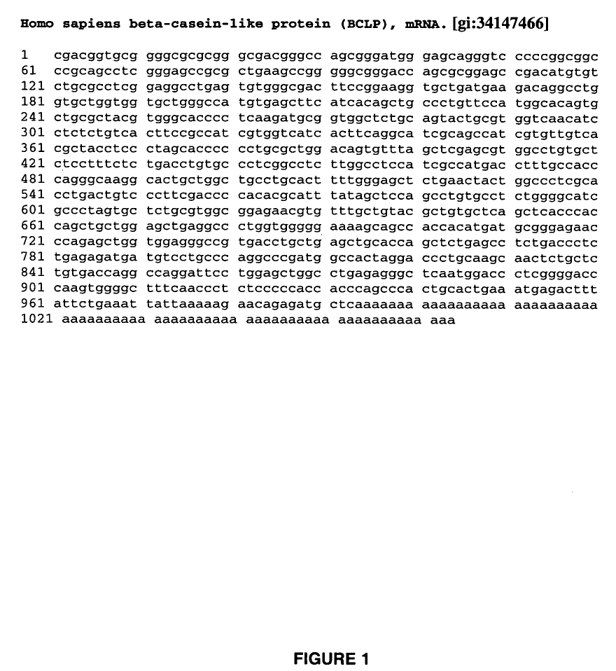

FIG. 2 depicts the amino acid sequence of the BCLP polypeptide (SEQ ID NO: 2) encoded by the polynucleotide of FIG. 1.

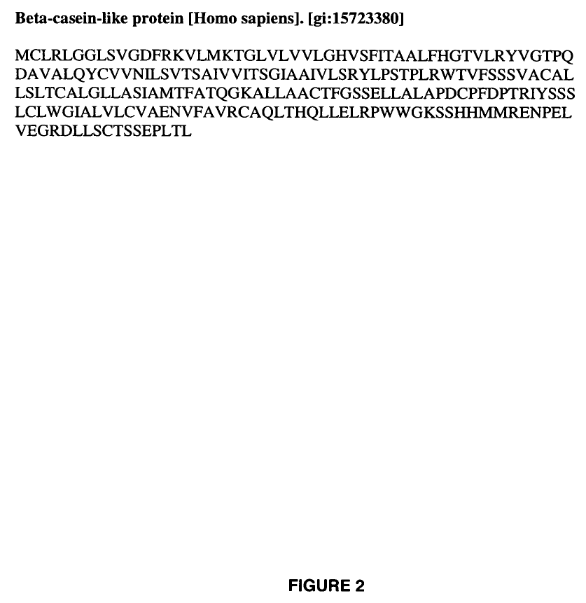

FIG. 3 shows a CLUSTAL V alignment of the BCLP polypeptide isoforms A and B (SEQ ID NO: 2), C (SEQ ID NO: 12), D (SEQ ID NO: 13), E and F (SEQ ID NO: 14), G (SEQ ID NO: 15), H (SEQ ID NO: 16), and I (SEQ ID NO: 17), wherein A=Alanine, C=Cysteine, D=Aspartic Acid, E=Glutamic Acid, F=Phenylalanine, G=Glycine, H=Histidine, I=lsoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

FIG. 4 shows the expression of BCLP mRNA derived from colon tumors and tissue adjacent to the colon tumors relative to the expression of BCLP in healthy organs.

### 4. DETAILED DESCRIPTION OF THE INVENTION

The present invention relates to methods of targeting cancer cells of the colon, breast, lung, ovary, pancreas, prostate, and melanoma cells that express BCLP using targeting elements, such as BCLP polypeptides, nucleic acids encoding BCLP, anti-BCLP antibodies, binding polypeptides, peptides, and small molecules, including fragments or other modifications of any of these elements.

The present invention provides a novel approach for diagnosing and treating cancer of the colon, breast, lung, ovary, pancreas, prostate, and melanoma cells, as well as disorders associated with BCLP-expressing cells. The method comprises administering an effective dose of targeting preparations including preparations that comprise a BCLP antigen, or antigen presenting cells, or pharmaceutical compositions comprising the targeting elements, BCLP polypeptides, nucleic acids encoding BCLP, anti-BCLP antibodies, or binding polypeptides, peptides, and small molecules described below. Targeting of BCLP on the cell membranes of BCLP-expressing cells is expected to inhibit the growth of or destroy such cells. An effective dose will be the amount of such targeting BCLP preparations necessary to target BCLP on the cell membrane and inhibit the growth of or destroy the BCLP-expressing cells and/or metastasis.

A further embodiment of the present invention is to enhance the effects of therapeutic agents and adjunctive agents used to treat and manage disorders associated with BCLP-expressing cells, by administering BCLP preparations with therapeutic and adjuvant agents commonly used to treat such disorders. Chemotherapeutic agents useful in treating neoplastic disease and antiproliferative agents and drugs used for immunosuppression include alkylating agents, such as nitrogen mustards, alkyl sulfonates, nitrosoureas, triazenes; antimetabolites, such as folic acid analogs, pyrimidine analogs, and purine analogs; natural products, such as vinca alkaloids, epipodophyllotoxins, antibiotics, and enzymes; miscellaneous agents such as polatinum coordination complexes, substituted urea, methyl hydrazine derivatives, and adrenocortical suppressant; and hormones and antagonists, such as adrenocorticosteroids, progestins, estrogens, androgens, and anti-estrogens (Calebresi and Parks, pp. 1240-1306 in, Eds. A. G Goodman, L. S. Goodman, T. W. Rall, and F. Murad, The Pharmacological Basis of Therapeutics, Seventh Edition, MacMillan Publishing Company, New York, (1985), incorporated herein by reference in their entirety).

Adjunctive therapy used in the management of such disorders includes, for example, radiosensitizing agents, coupling of antigen with heterologous proteins, such as globulin or beta-galactosidase, or inclusion of an adjuvant during immunization.

High doses may be required for some therapeutic agents to achieve levels to effectuate the target response, but may often be associated with a greater frequency of dose-related adverse effects. Thus, combined use of the therapeutic methods of the present invention with agents commonly used to treat BCLP protein-related colon cancer allows the use of relatively lower doses of such agents resulting in a lower frequency of adverse side effects associated with long-term administration of the conventional therapeutic agents. Thus another indication for the therapeutic methods of this invention is to reduce adverse side effects associated with conventional therapy of colon cancer associated with BCLP-expressing cells.

### 4.1 TARGETING OF BCLP

Beta Casein Like Protein (BCLP) is an antigen that is associated with uterine cancers, and exhibits immunological characteristics similar to those of bovine beta-casein (Horimoto et al., Asia Oceania J Obstet Gynacol 20:321-330 (1994); Suzuki et al., Cancer Lett 124:165-171 (1998)); Baba et al., Biochem Biophys Res commun 284:340-345 (2001), herein incorporated by reference in their entirety).

BCLP was first recognized by a monoclonal antibody, 1 C5, that was generated by immunization with CAC-1 human cervical adenocarcinoma cells (Koizumi et al., Cancer Res 48:6565-6572 (1988), herein incorporated by reference). Subsequently, the cDNA (SEQ ID NO: 1; Accession no. gi: 34147466) that encodes a 222 amino acid BCLP protein (SEQ ID NO: 2) was isolated from a library derived from the CAC-1 cells (Suzuki et al., Cancer Lett 124:165-171 (1998); Baba et al., Biochem Biophys Res commun 284:340-345 (2001), herein incorporated by reference in their entirety).

BCLP is expressed at high levels in cell lines derived from uterine cervical adenocarcinomas (Suzuki et al., Cancer Lett 124:165-171 (1998); Baba et al., Biochem Biophys Res commun 284:340-345 (2001) The expression of BCLP mRNA can also be detected in the blood of patients with gyneacologic malignancies, and the expression seems to correlate with the recurrence of the uterine cancers (Baba et al., Anticancer Res 21:2547-2552 (2001)).

While the function of BCLP is not known, BCLP increases cell area, and decreases cell growth rate and cell attachment of cells that are stably transfected with a vector containing BCLP cDNA. Thus, BCLP may affect cell morphology, and regulate the growth pattern of a tumor (Baba et al., Biochem Biophys Res commun 284:340-345 (2001), herein incorporated by reference).

BCLP is encoded by the CAC-1 gene, which is also known as 1_{—}32785843, maps on chromosome 1 at 1p35-p34. The CAC-1 gene may produce 9 different transcripts by alternative splicing (SEQ ID NOs: 3, 4, 5, 6, 7, 8, 9, 10, and 11) that correspond to mRNAs A-I, which altogether encode 7 different predicted protein isoforms. There are 3 probable alternative promoters, and 3 non overlapping alternative last exons. The transcripts appear to differ by truncation of the N-terminus, truncation of the C terminus, presence or absence of 2 cassette exons, common exons with different boundaries, because an internal intron is not always spliced out. Polynucleotides A and B (SEQ ID NOs: 3 and 4, respectively) encode the 222 amino acid polypeptide of SEQ ID NO: 2; polynucleotide C (SEQ ID NO: 5) encodes a 189 amino acid polypeptide of SEQ ID NO: 12; polynucleotide D (SEQ ID NO: 6) encodes a 169 amino acid polypeptide of SEQ ID NO: 13; polynucleotides E and F (SEQ ID NOs: 7 and 8, respectively) encode the 156 amino acid polypeptide of SEQ ID NO: 14, polynucleotide G (SEQ ID NO: 9) encodes a 142 amino acid polypeptide of SEQ ID NO: 15; polynucleotide H (SEQ ID NO: 10) encodes a 143 amino acid polypeptide of SEQ ID NO: 16; and polynucleotide I (SEQ ID NO: 11) encodes a 105 amino acid polypeptide of SEQ ID NO: 17.

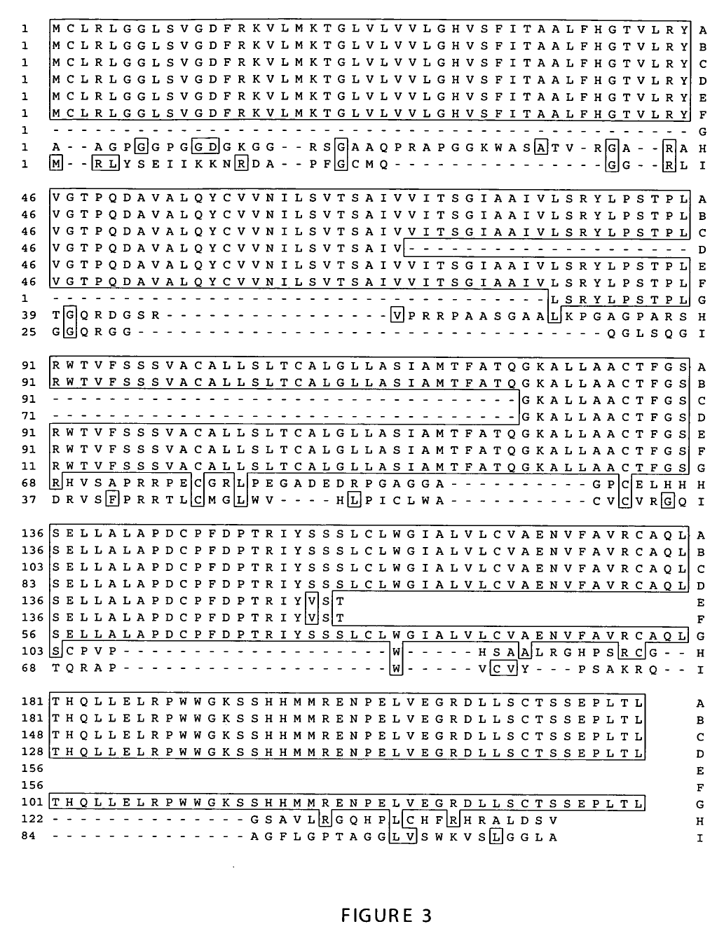

The information pertaining to the polynucleotides and isoforms of BCLP is publicly available at the National Center for Biological Information Web Site using AceView: mRNA CAC-1, at ncbi.nlm.nih.gov?IEB/Research/Acembly/av.cgi?db=human&C=Gene&I=CAC-1. The information from AceView is herein incorporated by reference. A Clustal V alignment of the 7 proteins isoforms encoded by the 9 transcripts is shown in FIG. 3.

BCLP herein refers to a BCLP protein that comprises a polypeptide that is encoded by a polynucleotide that can be amplified by RT-PCR using the primers of SEQ ID NOs: 20 and 21. The primers of SEQ ID NOs: 20 and 21 can be used in RT-PCR to amplify the polynucleotide of SEQ ID NO: 19, which encodes the polypeptide of SEQ ID NO: 18. The primers of SEQ ID NOs: 20 and 21 can be used to detect the presence of mRNA that encodes the BCLP polypeptides of SEQ ID NOs: 2, 12, 13, and 15.

Co-owned and copending U.S. patent application Ser. No. 09/799,451, filed Mar. 5, 2001, entitled “Novel Nucleic Acids and Polypeptides” discloses a BCLP polypeptide that is identical to that of SEQ ID NO: 2, or isoforms A and B, and a polypeptide that is identical to that of isoform C, herein SEQ ID NO: 12. The polynucleotides encoding the BCLP polypeptides are also disclosed in the U.S. Patent Application.

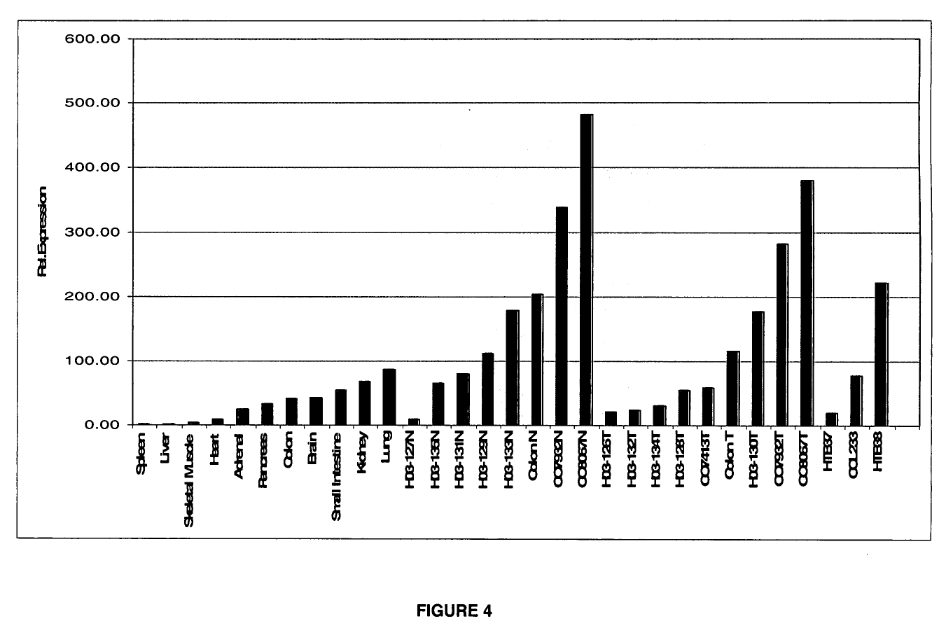

Applicants have shown that BCLP mRNA is expressed at low levels in various healthy organs including lung, kidney, small intestine, brain, colon, pancreas and adrenal gland, while its expression is barely detectable in heart and skeletal muscle, and is absent in liver and spleen. In addition, Applicants have discovered that BCLP mRNA is expressed at high levels in colon tumors and in tissue adjacent to the colon tumors when compared to the expression of BCLP in healthy organs (FIG. 4). The overexpression of mRNA in colon tumors is consistent with the overexpression of BCLP protein in colon tumors (Example 3). In addition, BCLP protein is overexpressed in cancer of the breast, lung, ovary, pancreas, prostate and skin (Example 3; Table 1). The restricted pattern of expression of BCLP indicates that BCLP is an antigen that is suitable for a variety of therapeutic strategies for targeting tumors of the colon, breast, lung, ovary, pancreas, prostate and skin.

### 4.2 DEFINITIONS

The term “colon cancer” herein refers to sporadic colorectal cancers and hereditary colorectal cancers including familial colorectal cancer, HNPCC (Hereditary Non Polyposis Colorectal Cancer), FAP (Familial Adenomatous Polyposis), Juvenile Polyposis, Gardner's syndrome, Turcot's syndrome, and Peutz-Jeghers syndrome. The histologic types of colon cancer include adenocarcinoma, mucinous adenocarcinoma, signet ring adenocarcinoma, scirrhous tumors, and neuroendocrine tumors.

The term “melanoma” refers to cancers of the skin, including melanomas, metastatic melanomas, melanomas derived from either melanocytes or melanocytes related nevus cells, melanocarcinomas, melanoepitheliomas, melanosarcomas, melanoma in situ, superficial spreading melanoma, nodular melanoma, lentigo maligna melanoma, acral lentiginous melanoma, invasive melanoma or familial atypical mole and melanoma (FAM-M) syndrome. Such melanomas in mammals may be caused by, chromosomal abnormalities, degenerative growth and developmental disorders, mitogenic agents, ultraviolet radiation (UV), viral infections, inappropriate tissue expression of a gene, alterations in expression of a gene, or carcinogenic agents. The aforementioned melanomas can be diagnosed, assessed or treated by methods described in the present application.

The term “lung cancer” refers to small cell lung cancer (SCLC), which includes small cell carcinoma, mixed small cell/large cell carcinoma, and combined small cell carcinoma (small cell lung cancer combined with neoplastic squamous and/or glandular components), and to non-small cell lung cancer (NSCLC), which includes squamous cell carcinoma, adenocarcinoma and large cell carcinoma.

The term “breast cancer” herein refers to invasive breast cancer that includes intraductal and lobular carcinoma in situ; non-invasive breast cancer that includes invasive ductal carcinoma, with or without a predominant intraductal component, invasive lobular carcinoma, mucinous carcinoma, medullary carcinoma, papillary carcinoma, tubular carcinoma, adenoid cystic carcinoma, secretory Ouvenile) carcinoma, apocrine carcinoma, carcinoma with metaplasia, squamous type, spindle-cell type, cartilaginous and osseus type, and mixed type carcinomas; and Pagets disease of the nipple.

The term “pancreatic cancer” herein refers to malignant tumors of the pancreas including tumors that arise in the glandular duct of the pancreas, and malignancies that arise in islet cells.

The term “ovarian cancer” or “cancer of the ovary” herein refers to malignant ovarian tumors that may be epithelial, germ cell or stromal tumors of the ovary.

The term “prostate cancer” herein refers to prostatic adenocarcinoma, which is a malignant tumour of glandular origin in the prostate.

The term “fragment” of a nucleic acid refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. Preferably the fragment comprises a sequence substantially similar to a portion of SEQ ID NO: 1. A polypeptide “fragment ” is a stretch of amino acid residues of at least about 5 amino acids, preferably at least about 7 amino acids, more preferably at least about 9 amino acids and most preferably at least about 17 or more amino acids. The peptide preferably is not greater than about 200 amino acids, more preferably less than 150 amino acids and most preferably less than 100 amino acids. Preferably the peptide is from about 5 to about 200 amino acids. To be active, any polypeptide must have sufficient length to display biological and/or immunological activity. The term “immunogenic” refers to the capability of the natural, recombinant or synthetic BCLP-like peptide, or any peptide thereof, to induce a specific immune response in appropriate animals or cells and to bind with specific antibodies.

The term “BCLP antigen” refers to a molecule that when introduced into an animal is capable of stimulating an immune response in said animal specific to the BCLP polypeptide or fragment thereof, of the present invention.

The term “variant” (or “analog”) refers to any polypeptide differing from naturally occurring polypeptides by amino acid insertions, deletions, and substitutions, created using, e g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

The term “stringent” is used to refer to conditions that are commonly understood in the art as stringent. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1× SSC/0.1% SDS at 68° C.), and moderately stringent conditions (i.e., washing in 0.2× SSC/0.1% SDS at 42° C.). Other exemplary hybridization conditions are described herein in the examples.

In instances of hybridization of deoxyoligonucleotides, additional exemplary stringent hybridization conditions include washing in 6× SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

### 4.3 TARGETING USING BCLP ANTIGENS

Use of a tumor antigen in a composition for generating cellular and humoral immunity for the purpose of anti-cancer therapy is well known in the art. For example, one type of tumor-specific composition uses purified idiotype protein isolated from tumor cells, coupled to keyhole limpet hemocyanin (KLH) and mixed with adjuvant for injection into patients with low-grade follicular lymphoma (Hsu, et al., Blood 89: 3129-3135 (1997), herein incorporated by reference in its entirety). U.S. Pat. No. 6,312,718, herein incorporated by reference in its entirety, describes methods for inducing immune responses against malignant B cells, in particular lymphoma, chronic lymphocytic leukemia, and multiple myeloma. One embodiment of the present invention provides a composition that comprises the BCLP antigen, for example the BCLP polypeptide of SEQ ID NO: 2, the extracellular portion or fragment thereof, to target BCLP-expressing cells by stimulating the immune system against BCLP. The methods described therein utilize vaccines that include liposomes having (1) at least one B-cell malignancy-associated antigen, (2) IL-2 alone, or in combination with at least one other cytokine or chemokine, and (3) at least one lipid molecule. Methods of targeting BCLP typically employ a BCLP polypeptide, including fragments, analogs and variants.

As another example, dendritic cells, one type of antigen-presenting cell, can be used in a cellular vaccine in which the dendritic cells are isolated from the patient, co-cultured with tumor antigen and then reinfused as a cellular vaccine (Hsu, et al., Nat Med. 2:52-58 (1996), herein incorporated by reference in its entirety).

Combining this vaccine therapy with other types of therapeutic agents in treatments such as chemotherapy or radiotherapy is also contemplated.

### 4.4 TARGETING USING NUCLEIC ACIDS

### 4.4.1 Direct Delivery of Nucleic Acids

In some embodiments, a nucleic acid encoding BCLP (for example, SEQ ID NO: 1), or encoding a fragment, analog or variant thereof, within a recombinant vector is utilized. Such methods are known in the art. For example, immune responses can be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002), herein incorporated by reference in its entirety). BCLP viral vectors are particularly useful for delivering nucleic acids encoding BCLP of the invention to cells. Examples of vectors include those derived from influenza, adenovirus, vaccinia, herpes symplex virus, fowlpox, vesicular stomatitis virus, canarypox, poliovirus, adeno-associated virus, and lentivirus and sindbus virus. Of course, non-viral vectors, such as liposomes or even naked DNA, are also useful for delivering nucleic acids encoding BCLP of the invention to cells.

Combining this type of therapy with other types of therapeutic agents or treatments such as chemotherapy or radiation is also contemplated.

### 4.4.2 BCLP Nucleic Acids Expressed in Cells

In some embodiments, a vector comprising a nucleic acid encoding the BCLP polypeptide (including a fragment, analog or variant) is introduced into a cell, such as a dendritic cell or a macrophage. When expressed in an antigen-presenting cell (APC), the BCLP cell surface antigens are presented to T cells eliciting an immune response against BCLP. Such methods are also known in the art. Methods of introducing tumor antigens into APCs and vectors useful therefore are described in U.S. Pat. No. 6,300,090, herein incorporated by reference in its entirety. The vector encoding BCLP may be introduced into the APCs in vivo. Alternatively, APCs are loaded with BCLP or a nucleic acid encoding BCLP ex vivo and then introduced into a patient to elicit an immune response against BCLP. In another alternative, the cells presenting BCLP antigen are used to stimulate the expansion of anti-BCLP cytotoxic T lymphocytes (CTL) ex vivo followed by introduction of the stimulated CTL into a patient. (U.S. Pat. No. 6,306,388, herein incorporated by reference in its entirety).

Combining this type of therapy with other types of therapeutic agents or treatments such as chemotherapy or radiation is also contemplated.

### 4.4.3 Antisense Nucleic Acids

Another aspect of the invention pertains to isolated antisense nucleic acid molecules that can hybridize to, or are complementary to, the nucleic acid molecule comprising the BCLP nucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire BCLP coding strand, or to only a portion thereof. Nucleic acid molecules encoding fragments, homologs, derivatives and analogs of a BCLP or antisense nucleic acids complementary to a BCLP nucleic acid sequence of are additionally provided.

In one embodiment, an antisense nucleic acid molecule is antisense to a “coding region” of the coding strand of a nucleotide sequence encoding a BCLP protein. The term “coding region” refers to the region of the nucleotide sequence comprising codons which are translated into amino acid residues. In another embodiment, the antisense nucleic acid molecule is antisense to a “conceding region” of the coding strand of a nucleotide sequence encoding the BCLP protein. The term “conceding region” refers to 5′ and 3′ sequences which flank the coding region that are not translated into amino acids (i.e., also referred to as 5′ and 3′ untranslated regions).

Given the coding strand sequences encoding the BCLP protein disclosed herein, antisense nucleic acids of the invention can be designed according to the rules of Watson and Crick or Hoogsteen base pairing. The antisense nucleic acid molecule can be complementary to the entire coding region of BCLP mRNA, but more preferably is an oligonucleotide that is antisense to only a portion of the coding or noncoding region of BCLP mRNA. For example, the antisense oligonucleotide can be complementary to the region surrounding the translation start site of BCLP mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a BCLP protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res. 15: 6625-6641 (1987). The antisense nucleic acid molecule can also comprise a 2′-o-methylribonucleotide (see, e.g., Inoue, et al., Nucl. Acids Res. 15: 6131-6148 (1987)) or a chimeric RNA-DNA analogue (see, e.g., Inoue, et al., FEBS Lett. 215: 327-330 (1987), all of which are herein incorporated by reference in their entirety.

### 4.4.4 Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of an mRNA. A ribozyme having specificity for a nucleic acid of the invention can be designed based upon a nucleotide sequence of a DNA disclosed herein (i.e., SEQ ID NO: 1). For example, a derivative of Tetrahymena L-19 IVS RNA can be constructed in which the nucleotide sequence of the active site is complementary to the nucleotide sequence to be cleaved in a mRNA. See, e.g., Cech et al. U.S. Pat. No. 4,987,071; and Cech et al. U.S. Pat. No. 5,116,742. Alternatively, mRNA of the invention can be used to select a catalytic RNA having a specific ribonuclease activity from a pool of RNA molecules. See, e.g., Bartel et al., (1993) Science 261:1411-1418.

Alternatively, gene expression can be inhibited by targeting nucleotide sequences complementary to the regulatory region (e.g., promoter and/or enhancers) to form triple helical structures that prevent transcription of the gene in target cells. See generally, Helene. (1991) Anticancer Drug Des. 6: 569-84; Helene. et al. (1992) Ann. N. Y. Acad. Sci. 660:27-36; and Maher (1992) Bioassays 14: 807-15.

In various embodiments, the nucleic acids of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of the invention can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of the invention can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/09810) or the blood-brain barrier (see, e.g., PCT Publication No. W089/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

### 4.4.5 Gene Therapy

Mutations in the polynucleotides of the invention gene may result in loss of normal function of the encoded protein. The invention thus provides gene therapy to restore normal activity of the polypeptides of the invention; or to treat disease states involving polypeptides of the invention. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, 392(Suppl):25-20 (1998). For additional reviews of gene therapy technology see Friedmann, Science, 244: 1275-1281 (1989); Verma, Scientific American: 68-84 (1990); and Miller, Nature, 357: 455-460 (1992), all of which are herein incorporated by reference in their entirety. Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. Treated cells can then be introduced in vivo for therapeutic purposes. Alternatively, it is contemplated that in other human disease states, preventing the expression of or inhibiting the activity of polypeptides of the invention will be useful in treating the disease states. It is contemplated that antisense therapy or gene therapy could be applied to negatively regulate the expression of polypeptides of the invention.

Other methods inhibiting expression of a protein include the introduction of antisense molecules to the nucleic acids of the present invention, their complements, or their translated RNA sequences, by methods known in the art. Further, the polypeptides of the present invention can be inhibited by using targeted deletion methods, or the insertion of a negative regulatory element such as a silencer, which is tissue specific.

The present invention still further provides cells genetically engineered in vivo to express the polynucleotides of the invention, wherein such polynucleotides are in operative association with a regulatory sequence heterologous to the host cell which drives expression of the polynucleotides in the cell. These methods can be used to increase or decrease the expression of the polynucleotides of the present invention.

Knowledge of DNA sequences provided by the invention allows for modification of cells to permit, increase, or decrease, expression of endogenous polypeptide. Cells can be modified (e.g., by homologous recombination) to provide increased polypeptide expression by replacing, in whole or in part, the naturally occurring promoter with all or part of a heterologous promoter so that the cells express the protein at higher levels. The heterologous promoter is inserted in such a manner that it is operatively linked to the desired protein encoding sequences. See, for example, PCT International Publication No. WO 94/12650, PCT International Publication No. WO 92/20808, and PCT International Publication No. WO 91/09955, all of which are incorporated by reference in their entirety. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the desired protein coding sequence, amplification of the marker DNA by standard selection methods results in co-amplification of the desired protein coding sequences in the cells.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequences include polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

### 4.5 ANTI-BCLP ANTIBODIES

Targeting of BCLP-expressing cells involves the administration of components of the immune system, such as antibodies, antibody fragments, or primed cells of the immune system against the target. Methods of immunotargeting cancer cells using antibodies or antibody fragments are well known in the art. U.S. Pat. No. 6,306,393 describes the use of anti-CD22 antibodies in the immunotherapy of B-cell malignancies, and U.S. Pat. No. 6,329,503 describes immunotargeting of cells that express serpentine transmembrane antigens (both U.S. patents are herin incorporated by reference in their entirety).

BCLP antibodies (including humanized or human monoclonal antibodies or fragments or other modifications thereof, optionally conjugated to cytotoxic agents) may be introduced into a patient such that the antibody binds to BCLP expressed by cancer cells and mediates the destruction of the cells and the tumor and/or inhibits the growth of the cells or the tumor. Without intending to limit the disclosure, mechanisms by which such antibodies can exert a therapeutic effect may include complement-mediated cytolysis, antibody-dependent cellular cytotoxicity (ADCC), modulating the physiologic function of BCLP, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis. BCLP antibodies conjugated to toxic or therapeutic agents, such as radioligands or cytosolic toxins, may also be used therapeutically to deliver the toxic or therapeutic agent directly to BCLP-bearing tumor cells. Prodrug-activating enzymes may be conjugated to BCLP antibodies for use in antibody-directed enzyme prodrug therapy (ADEPT).

BCLP antibodies may be used to suppress the immune system in patients receiving organ transplants or in patients with autoimmune diseases such as arthritis. Healthy immune cells would be targeted by these antibodies leading their death and clearance from the system, thus suppressing the immune system.

BCLP antibodies may be used as antibody therapy for solid tumors which express BCLP. Cancer immunotherapy using antibodies provides a novel approach to treating cancers associated with cells that specifically express BCLP. Cancer immunotherapy using antibodies has been previously described for other types of cancer, including but not limited to colon cancer (Arlen et al., Crit. Rev. Immunol. 18:133-138 (1998)), multiple myeloma (Ozaki et al., Blood 90:3179-3186 (1997); Tsunenari et al., Blood 90:2437-2444 (1997)), gastric cancer (Kasprzyk et al., Cancer Res. 52:2771-2776 (1992)), B cell lymphoma (Funakoshi et al., J. Immunother. Emphasisi Tumor Immunol 19:93-101 (1996)), leukemia (Zhong et al., Leuk. Res. 20:581-589 (1996)), colorectal cancer (Moun et al., Cancer Res. 54:6160-6166 (1994); Velders et al., Cancer Res. 55:4398-4403 (1995)), and breast cancer (Shepard et al., J. Clin. Immunol. 11:117-127 (1991), all of the above listed references are herein incorporated by reference in their entirety).

Although BCLP antibody therapy may be useful for all stages of the foregoing cancers, antibody therapy may be particularly appropriate in advanced or metastatic cancers. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with BCLP antibody with tumors resistant to chemotherapeutic agents might induce sensitivity and responsiveness to these agents in combination.

Prior to anti-BCLP immunotargeting, a patient may be evaluated for the presence and level of BCLP expression by the cancer cells, preferably using immunohistochemical assessments of tumor tissue, quantitative BCLP imaging, quantitative RT-PCR, or other techniques capable of reliably indicating the presence and degree of BCLP expression. For example, a blood or biopsy sample may be evaluated by immunohistochemical methods to determine the presence of BCLP-expressing cells or to determine the extent of BCLP expression on the surface of the cells within the sample. Methods for immunohistochemical analysis of tumor tissues or released fragments of BCLP in the serum are well known in the art.

Anti-BCLP antibodies useful in treating cancers include those, which are capable of initiating a potent immune response against the tumor and those, which are capable of direct cytotoxicity. In this regard, anti-BCLP mAbs may elicit tumor cell lysis by either complement-mediated or ADCC mechanisms, both of which require an intact Fc portion of the immunoglobulin molecule for interaction with effector cell Fc receptor sites or complement proteins. In addition, anti-BCLP antibodies that exert a direct biological effect on tumor growth are useful in the practice of the invention. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-BCLP antibody exerts an anti-tumor effect may be evaluated using any number of in vitro assays designed to determine ADCC, ADMMC, complement-mediated cell lysis, and so forth, as is generally known in the art.

The anti-tumor activity of a particular anti-BCLP antibody, or combination of anti-BCLP antibody, may be evaluated in vivo using a suitable animal model. For example, xenogenic lymphoma cancer models wherein human lymphoma cells are introduced into immune compromised animals, such as nude or SCID mice. Efficacy may be predicted using assays, which measure inhibition of tumor formation, tumor regression or metastasis, and the like.

It should be noted that the use of murine or other non-human monoclonal antibodies, human/mouse chimeric mAbs may induce moderate to strong immune responses in some patients. In the most severe cases, such an immune response may lead to the extensive formation of immune complexes, which, potentially, can cause renal failure. Accordingly, preferred monoclonal antibodies used in the practice of the therapeutic methods of the invention are those which are either fully human or humanized and which bind specifically to the target BCLP antigen with high affinity but exhibit low or no antigenicity in the patient.

The method of the invention contemplates the administration of single anti-BCLP monoclonal antibodies (mAbs) as well as combinations, or “cocktails”, of different mAbs. Two or more monoclonal antibodies that bind to BCLP may provide an improved effect compared to a single antibody. Alternatively, a combination of an anti-BCLP antibody with an antibody that binds a different antigen may provide an improved effect compared to a single antibody. Such mAb cocktails may have certain advantages inasmuch as they contain mAbs, which exploit different effector mechanisms or combine directly cytotoxic mAbs with mAbs that rely on immune effector functionality. Such mAbs in combination may exhibit synergistic therapeutic effects. In addition, the administration of anti-BCLP mAbs may be combined with other therapeutic agents, including but not limited to various chemotherapeutic agents, androgen-blockers, and immune modulators (e.g., IL-2, GM-CSF). The anti-BCLP mAbs may be administered in their “naked” or unconjugated form, or may have therapeutic agents conjugated to them. Additionally, bispecific antibodies may be used. Such an antibody would have one antigenic binding domain specific for BCLP and the other antigenic binding domain specific for another antigen (such as CD20 for example). Finally, Fab BCLP antibodies or fragments of these antibodies (including fragments conjugated to other protein sequences or toxins) may also be used as therapeutic agents.

Antibodies that specifically bind BCLP are useful in compositions and methods for immunotargeting cells expressing BCLP and for diagnosing a disease or disorder wherein cells involved in the disorder express BCLP. Such antibodies include monoclonal and polyclonal antibodies, single chain antibodies, chimeric antibodies, bifunctional/bispecific antibodies, humanized antibodies, human antibodies, and complementary determining region (CDR)-grafted antibodies, including compounds that include CDR and/or antigen-binding sequences, which specifically recognize BCLP. Antibody fragments, including Fab, Fab′, F(ab′)₂, and Fᵥ, are also useful.

BCLP polypeptides can be used to immunize animals to obtain polyclonal and monoclonal antibodies that specifically react with BCLP. Such antibodies can be obtained using either the entire protein (for example SEQ ID NO: 2) or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides have been previously described (Merrifield, J. Amer. Chem. Soc. 85, 2149-2154 (1963); Krstenansky, et al., FEBS Lett. 211: 10 (1987), both of which are incorporated by reference in their entirety). Techniques for preparing polyclonal and monoclonal antibodies as well as hybridomas capable of producing the desired antibody have also been previously disclosed (Campbell, Monoclonal Antibodies Technology: Laboratory Techniques in Biochemistry and Molecular Biology, Elsevier Science Publishers, Amsterdam, The Netherlands (1984); St. Groth, et al., J. Immunol. 35:1-21 (1990); Kohler and Milstein, Nature 256:495-497 (1975)), the trioma technique, the human B-cell hybridoma technique (Kozbor, et al., Immunology Today 4:72 (1983); Cole, et al., in, Monoclonal Antibodies and Cancer Therapy, Alan R. Liss, Inc., pp. 77-96 (1985), all of which are incorporated by reference in their entirety).

Any animal capable of producing antibodies can be immunized with a BCLP peptide or polypeptide. Methods for immunization include subcutaneous or intraperitoneal injection of the polypeptide. The amount of the BCLP peptide or polypeptide used for immunization depends on the animal that is immunized, antigenicity of the peptide and the site of injection. The BCLP peptide or polypeptide used as an immunogen may be modified or administered in an adjuvant in order to increase the protein's antigenicity. Methods of increasing the antigenicity of a protein are well known in the art and include, but are not limited to, coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell that produces an antibody with the desired characteristics. These include screening the hybridomas with an ELISA assay, Western blot analysis, or radioimmunoassay (Lutz, et al., Exp. Cell Res. 175:109-124 (1988), herein incorporated by reference in its entirety). Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984), herein incorporated by reference in its entirety). Techniques described for the production of single chain antibodies can be adapted to produce single chain antibodies to BCLP (U.S. Pat. No. 4,946,778, herein incorporated by reference in its entirety).

For polyclonal antibodies, antibody-containing antiserum is isolated from the immunized animal and is screened for the presence of antibodies with the desired specificity using one of the above-described procedures.

Because antibodies from rodents tend to elicit strong immune responses against the antibodies when administered to a human, such antibodies may have limited effectiveness in therapeutic methods of the invention. Methods of producing antibodies that do not produce a strong immune response against the administered antibodies are well known in the art. For example, the anti-BCLP antibody can be a nonhuman primate antibody. Methods of making such antibodies in baboons are disclosed in PCT publication No. WO 91/11465 and Losman et al., Int. J. Cancer 46:310-314 (1990), both of which are herein incorporated by reference in their entirety. In one embodiment, the anti-BCLP antibody is a humanized monoclonal antibody. Methods of producing humanized antibodies have been previously described. (U.S. Pat. Nos. 5,997,867 and 5,985,279, Jones et al., Nature 321:522 (1986); Riechmann et al., Nature 332:323(1988); Verhoeyen et al., Science 239:1534-1536 (1988); Carter et al., Proc. Nat'l Acad. Sci. USA 89:4285-4289 (1992); Sandhu, Crit. Rev. Biotech. 12:437-462 (1992); and Singer, et al., J. Immun. 150:2844-2857 (1993), all of which are herein incorporated by reference in their entirety). In another embodiment, the anti-BCLP antibody is a human monoclonal antibody. Humanized antibodies are produced by transgenic mice that have been engineered to produce human antibodies. Hybridomas derived from such mice will secrete large amounts of human monoclonal antibodies. Methods for obtaining human antibodies from transgenic mice are described in Green , et al., Nature Genet. 7:13-21(1994), Lonberg, et al., Nature 368:856 (1994), and Taylor, et al., Int. Immun. 6:579 (1994), all of which are herein incorporated by reference in their entirety.

The present invention also includes the use of anti-BCLP antibody fragments. Antibody fragments can be prepared by proteolytic hydrolysis of an antibody or by expression in E. coli of the DNA coding for the fragment. Antibody fragments can be obtained by pepsin or papain digestion of whole antibodies. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment directly. These methods have been previously described (U.S. Pat. Nos. 4,036,945 and 4,331,647, Nisonoff, et al., Arch Biochem. Biophys. 89:230 (1960); Porter, Biochem. J. 73:119 (1959), Edelman, et al., Meth. Enzymol. 1:422 (1967), all of which are herein incorporated by reference in their entirety). Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody. For example, Fv fragments comprise an association of V_{H} and V_{L} chains, which can be noncovalent (Inbar et al., Proc. Nat'l Acad. Sci. USA 69:2659 (1972), herein incorporated by reference in its entirety). Alternatively, the variable chains can be linked by an intermolecular disulfide bond or cross-linked by chemicals such as glutaraldehyde.

In one embodiment, the Fv fragments comprise V_{H} and V_{L} chains that are connected by a peptide linker. These single-chain antigen binding proteins (sFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing sFvs have been previously described (U.S. Pat. No. 4,946,778, Whitlow, et al., Methods: A Companion to Methods in Enzymology 2:97 (1991), Bird, et al., Science 242:423 (1988), Pack, et al., Bio/Technology 11:1271 (1993), all of which are herein incorporated by reference in their entirety).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick, et al., Methods: A Companion to Methods in Enymology 2:106 (1991); Courtenay-Luck, pp. 166-179 in, Monoclonal Antibodies Production, Engineering and Clinical Applications, Eds. Ritter et al., Cambridge University Press (1995); Ward, et al., pp. 137-185 in, Monoclonal Antibodies Principles and Applications, Eds. Birch et al., Wiley-Liss, Inc. (1995), all of which are herein incorporated by reference in their entirety).

The present invention further provides the above- described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Procedures for accomplishing such labeling have been previously disclosed (Sternberger, et al., J. Histochem. Cytochem. 18:315 (1970); Bayer, et al., Meth. Enzym. 62:308 (1979); Engval, et al., Immunol. 109:129 (1972); Goding, J. Immunol. Meth. 13:215 (1976), all of which are herein incorporated by reference in their entirety).

The labeled antibodies can be used for in vitro, in vivo, and in situ assays to identify cells or tissues in which BCLP is expressed. Furthermore, the labeled antibodies can be used to identify the presence of secreted BCLP in a biological sample, such as a blood, urine, saliva samples.

### 4.5.1 Antibody Conjugates

The present invention contemplates the use of “naked” anti-BCLP antibodies, as well as the use of antibody conjugates. Antibody conjugates can be prepared by indirectly conjugating a therapeutic agent such as a cytotoxic agent or a prodrug activating enzyme to an antibody component. Toxic moieties include, for example, plant toxins, such as abrin, ricin, modeccin, viscumin, pokeweed anti-viral protein, saporin, gelonin, momoridin, trichosanthin, barley toxin; bacterial toxins, such as Diptheria toxin, Pseudomonas endotoxin and exotoxin, Staphylococcal enterotoxin A; fungal toxins, such as a-sarcin, restrictocin; cytotoxic RNases, such as extracellular pancreatic RNases; DNase I (Pastan, et al., Cell 47:641 (1986); Goldenberg, Cancer Journal for Clinicians 44:43 (1994), herein incorporated by reference in their entirety), calicheamicin, and radioisotopes, such as ³²P, ⁶⁷CU, ⁷⁷As, ¹⁰⁵Rh, ¹⁰⁹Pd, ¹¹¹Ag, ¹²¹Sn, ¹³¹I, ¹⁶⁶Ho, ¹⁷⁷Lu, ¹⁸⁶Re, ¹⁸⁸Re, ¹⁹⁴Ir, ¹⁹⁹Au (Illidge, T. M. & Brock, S., Curr Pharm. Design 6: 1399 (2000), herein incorporated by reference in its entirety). In humans, clinical trials are underway utilizing a yttrium-90 conjugated anti-CD20 antibody for B cell lymphomas (Cancer Chemother Pharmacol48(Suppl 1):S91-S95 (2001), herein incorporated by reference in its entirety).

Enzyme proteins including prodrug-activating enzymes may be conjugated to an antibody for use in antibody-directed enzyme prodrug therapy (ADEPT), which has been developed to overcome the unwanted nonspecific toxicity associated with anticancer agents. There are two components to such therapy: and antibody-enzyme conjugate and an anticancer prodrug of low toxicity. The conjugate is administered first and accumulates predominantly at the tumor site through antibody binding to tumor-associated antigenic determinants. Once the conjugate has been cleared from the plasma, the prodrug is administered to the patient. Cleavage of the prodrug to generate the active cytotoxic agent by the enzyme component of the conjugate occurs selectively at the tumor site, and so leads to both enhanced efficacy of the anticancer agent and to reduced peripheral cytotoxicity (Xu and McLeod, Clin Cancer Res 7:3314-3324 (2001); Wentworth et al., Proc Natl Acad Sci 93:799-803 (1996), herein incorporated by reference in their entirety.) The enzyme can be a human protein that is absent or is expressed only at low concentrations in normal tissues, or the enzyme can be of non-human origin. The advantage of using an enzyme of human origin lies in avoiding or minimizing the immunogenic effect of an enzyme of non-human origin. When the enzyme is of non-human origin the immunoconjugate can be rendered less immunogenic by conjugating it to polyethylene glycol or other polymers, or it can be mutated. Examples of suitable enzymes are: carboxypeptidase G2, carboxypeptidase A, aminopeptidase, alkaline phsphatase, glycosidases, β-glucuronidase, penicillin amidase, β-lactamase, cytosine deaminase, nitroreductase, or mutant host enzymes including carboxypeptidase a and B, and ribonuclease (U.S. Pat. No. 6,339,070). Examples of prodrugs and enzymes that are suitable for ADEPT, and methods for the treatment of colon tumors using ADEPT are disclosed in U.S. Pat. Nos. 5,683,694; 5,632,990; 5,660,829; and 6,339,070, all of which are herein incorporated by reference in their entirety. It is contemplated that the anti-BCLP antibody is conjugated to a prodrug-activating enzyme for use in ADEPT therapy.

General techniques for preparing antibody conjugates have been previously described (U.S. Pat. Nos. 6,306,393 and 5,057,313, Shih, et al., Int. J. Cancer 41:832-839 (1988); Shih, et al., Int. J. Cancer46:1101-1106 (1990), all of which are herein incorporated by reference in their entirety). The general method involves reacting an antibody component having an oxidized carbohydrate portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form the final conjugate.

The carrier polymer is preferably an aminodextran or polypeptide of at least 50 amino acid residues, although other substantially equivalent polymer carriers can also be used. Preferably, the final immunoconjugate is soluble in an aqueous solution, such as mammalian serum, for ease of administration and effective targeting for use in therapy. Thus, solubilizing functions on the carrier polymer will enhance the serum solubility of the final immunoconjugate. In particular, an aminodextran will be preferred.

The process for preparing an inmmunoconjugate with an aminodextran carrier typically begins with a dextran polymer, advantageously a dextran of average molecular weight of about 10,000-100,000. The dextran is reacted with an oxidizing agent to affect a controlled oxidation of a portion of its carbohydrate rings to generate aldehyde groups. The oxidation is conveniently effected with glycolytic chemical reagents such as NaIO₄, according to conventional procedures. The oxidized dextran is then reacted with a polyamine, preferably a diamine, and more preferably, a mono- or polyhydroxy diamine. Suitable amines include ethylene diamine, propylene diamine, or other like polymethylene diamines, diethylene triamine or like polyamines, 1,3-diamino-2-hydroxypropane, or other like hydroxylated diamines or polyamines, and the like. An excess of the amine relative to the aldehyde groups of the dextran is used to ensure substantially complete conversion of the aldehyde functions to Schiff base groups. A reducing agent, such as NaBH₄, NaBH₃ CN or the like, is used to effect reductive stabilization of the resultant Schiff base intermediate. The resultant adduct can be purified by passage through a conventional sizing column or ultrafiltration membrane to remove cross-linked dextrans. Other conventional methods of derivatizing a dextran to introduce amine functions can also be used, e.g., reaction with cyanogen bromide, followed by reaction with a diamine.

The amninodextran is then reacted with a derivative of the particular drug, toxin, chelator, immunomodulator, boron addend, or other therapeutic agent to be loaded, in an activated form, preferably, a carboxyl-activated derivative, prepared by conventional means, e.g., using dicyclohexylcarbodiimide (DCC) or a water soluble variant thereof, to form an intermediate adduct. Alternatively, polypeptide toxins such as pokeweed antiviral protein or ricin A-chain, and the like, can be coupled to aminodextran by glutaraldehyde condensation or by reaction of activated carboxyl groups on the protein with amines on the aminodextran.

Chelators for radiometals or magnetic resonance enhancers are well-known in the art. Typical are derivatives of ethylenediaminetetraacetic acid (EDTA) and diethylenetriaminepentaacetic acid (DTPA). These chelators typically have groups on the side chain by which the chelator can be attached to a carrier. Such groups include, e.g., benzylisothiocyanate, by which the DTPA or EDTA can be coupled to the amine group of a carrier. Alternatively, carboxyl groups or amine groups on a chelator can be coupled to a carrier by activation or prior derivatization and then coupling, all by well-known means.

Boron addends, such as carboranes, can be attached to antibody components by conventional methods. For example, carboranes can be prepared with carboxyl functions on pendant side chains, as is well known in the art. Attachment of such carboranes to a carrier, e.g., aminodextran, can be achieved by activation of the carboxyl groups of the carboranes and condensation with amines on the carrier to produce an intermediate conjugate. Such intermediate conjugates are then attached to antibody components to produce therapeutically useful immunoconjugates, as described below.

A polypeptide carrier can be used instead of aminodextran, but the polypeptide carrier should have at least 50 amino acid residues in the chain, preferably 100-5000 amino acid residues. At least some of the amino acids should be lysine residues or glutamate or aspartate residues. The pendant amines of lysine residues and pendant carboxylates of glutamine and aspartate are convenient for attaching a drug, toxin, immunomodulator, chelator, boron addend or other therapeutic agent. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier and immunoconjugate.

Conjugation of the intermediate conjugate with the antibody component is effected by oxidizing the carbohydrate portion of the antibody component and reacting the resulting aldehyde (and ketone) carbonyls with amine groups remaining on the carrier after loading with a drug, toxin, chelator, immunomodulator, boron addend, or other therapeutic agent. Alternatively, an intermediate conjugate can be attached to an oxidized antibody component via amine groups that have been introduced in the intermediate conjugate after loading with the therapeutic agent. Oxidation is conveniently effected either chemically, e.g., with NaIO₄ or other glycolytic reagent, or enzymatically, e.g., with neuraminidase and galactose oxidase. In the case of an aminodextran carrier, not all of the amines of the aminodextran are typically used for loading a therapeutic agent. The remaining amines of aminodextran condense with the oxidized antibody component to form Schiff base adducts, which are then reductively stabilized, normally with a borohydride reducing agent.

Analogous procedures are used to produce other immunoconjugates according to the invention. Loaded polypeptide carriers preferably have free lysine residues remaining for condensation with the oxidized carbohydrate portion of an antibody component. Carboxyls on the polypeptide carrier can, if necessary, be converted to amines by, e.g., activation with DCC and reaction with an excess of a diamine.

The final immunoconjugate is purified using conventional techniques, such as sizing chromatography on Sephacryl S-300 or affinity chromatography using one or more BCLP epitopes.

Alternatively, immunoconjugates can be prepared by directly conjugating an antibody component with a therapeutic agent. The general procedure is analogous to the indirect method of conjugation except that a therapeutic agent is directly attached to an oxidized antibody component. It will be appreciated that other therapeutic agents can be substituted for the chelators described herein. Those of skill in the art will be able to devise conjugation schemes without undue experimentation.

As a further illustration, a therapeutic agent can be attached at the hinge region of a reduced antibody component via disulfide bond formation. For example, the tetanus toxoid peptides can be constructed with a single cysteine residue that is used to attach the peptide to an antibody component. As an alternative, such peptides can be attached to the antibody component using a heterobifunctional cross-linker, such as N-succinyl 3-(2-pyridyldithio) proprionate (SPDP) (Yu, et al., Int. J. Cancer 56:244 (1994), herein incorporated by reference in its entirety). General techniques for such conjugation have been previously described (Wong, Chemistry of Protein Conjugation and Cross-linking, CRC Press (1991); Upeslacis, et al., pp.187-230 in, Monoclonal Antibodies Principles and Applications, Eds. Birch et al., Wiley-Liss, Inc. (1995); Price, pp. 60-84 in, Monoclonal Antibodies: Production, Engineering and Clinical Applications Eds. Ritter, et al., Cambridge University Press (1995), all of which are herein incorporated by reference in their entirety).

As described above, carbohydrate moieties in the Fc region of an antibody can be used to conjugate a therapeutic agent. However, the Fc region may be absent if an antibody fragment is used as the antibody component of the immunoconjugate. Nevertheless, it is possible to introduce a carbohydrate moiety into the light chain variable region of an antibody or antibody fragment (Leung, et al., J. Immunol. 154:5919-5926 (1995); U.S. Pat. No. 5,443,953), both of which are herein incorporated by reference in their entirety. The engineered carbohydrate moiety is then used to attach a therapeutic agent.

In addition, those of skill in the art will recognize numerous possible variations of the conjugation methods. For example, the carbohydrate moiety can be used to attach polyethyleneglycol in order to extend the half-life of an intact antibody, or antigen-binding fragment thereof, in blood, lymph, or other extracellular fluids. Moreover, it is possible to construct a “divalent immunoconjugate” by attaching therapeutic agents to a carbohydrate moiety and to a free sulfhydryl group. Such a free sulfhydryl group may be located in the hinge region of the antibody component.

### 4.5.2 Antibody Fusion Proteins

When the therapeutic agent to be conjugated to the antibody is a protein, the present invention contemplates the use of fusion proteins comprising one or more anti-BCLP antibody moieties and an immunomodulator or toxin moiety. Methods of making antibody fusion proteins have been previously described (U.S. Pat. No. 6,306,393, herein incorporated by reference in its entirety). Antibody fusion proteins comprising an interleukin-2 moiety have also been previously disclosed (Boleti, et al., Ann. Oncol. 6:945 (1995), Nicolet, et al., Cancer Gene Ther. 2:161 (1995), Becker, et al., Proc. Nat'l Acad. Sci. USA 93:7826 (1996), Hank, et al., Clin. Cancer Res. 2:1951 (1996), Hu, et al., Cancer Res. 56:4998 (1996)all of which are herein incorporated by reference in their entirety). In addition, Yang, et al., Hum. Antibodies Hybridomas 6:129 (1995), herein incorporated by reference in its entirety, describe a fusion protein that includes an F(ab′)₂ fragment and a tumor necrosis factor alpha moiety.

Methods of making antibody-toxin fusion proteins in which a recombinant molecule comprises one or more antibody components and a toxin or chemotherapeutic agent also are known to those of skill in the art. For example, antibody-Pseudomonas exotoxin A fusion proteins have been described (Chaudhary, et al., Nature 339:394 (1989), Brinkmann, et al., Proc. Nat'l Acad. Sci. USA 88:8616 (1991), Batra, et al., Proc. Natl. Acad. Sci. USA 89:5867 (1992), Friedman, et al., J. Immunol. 150:3054 (1993), Wels, et al., Int. J. Can. 60:137 (1995), Fominaya et al., J. Biol. Chem. 271:10560 (1996), Kuan, et al., Biochemistry 35:2872 (1996), Schmidt, et al., Int. J. Can. 65:538 (1996), all of which are herein incorporated by reference in their entirety). Antibody-toxin fusion proteins containing a diphtheria toxin moiety have been described (Kreitman, et al., Leukemia 7:553 (1993), Nicholls, et al., J. Biol. Chem. 268:5302 (1993), Thompson, et al., J. Biol. Chem. 270:28037 (1995), and Vallera, et al., Blood 88:2342 (1996). Deonarain et al. (Tumor Targeting 1:177 (1995)), have described an antibody-toxin fusion protein having an RNase moiety, while Linardou, et al. (Cell Biophys. 24-25:243 (1994), all of which are herein incorporated by reference in their entirety), produced an antibody-toxin fusion protein comprising a DNase I component. Gelonin and Staphylococcal enterotoxin-A have been used as the toxin moieties in antibody-toxin fusion proteins (Wang, et al., Abstracts of the 209th ACS National Meeting, Anaheim, Calif., Apr. 2-6, 1995, Part 1, BIOT005; Dohlsten, et al., Proc. Nat'l Acad. Sci. USA 91:8945 (1994), both of which herein incorporated by reference in their entirety).

### 4.5.3 Fab Fragments and Single Chain Antibodies

According to the invention, techniques can be adapted for the production of single-chain antibodies specific to BCLP (see e.g., U.S. Pat. No. 4,946,778). In addition, methods can be adapted for the construction of F_{ab} expression libraries (see e.g., Huse, et al., Science 246:1275-1281 (1989)) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### 4.5.4 Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10, 3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121: 210 (1986).

According to another approach described in WO 96/27011, the interface between a pair of antibody molecules can be engineered to maximize the percentage of heterodimers that are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991). Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

### 4.5.5 Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### 4.5.6 Effector Functions Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176:1191-1195 (1992) and Shopes, J. Immunol., 148:2918-2922 (1992). Homodimeric antibodies with enhanced anti-tumor activity can also be prepared using heterobifunctional cross-linkers as described in Wolff et al. Cancer Research, 53:2560-2565 (1993). Alternatively, an antibody can be engineered that has dual Fc regions and can thereby have enhanced complement lysis and ADCC capabilities. See Stevenson et al., Anti-Cancer Drug Design, 3:219-230 (1989). 4.6 BCLP PEPTIDES

The BCLP peptide itself may be used to target toxins or radioisotopes to tumor cells in vivo. BCLP may be a homophilic adhesion protein which will bind to itself. In this case, the extracellular domain of BCLP, or a fragment of this domain, may be able to bind to BCLP expressed on colon cancer cells. This fragment may then be used as a means to deliver cytotoxic agents to BCLP expressing colon cancer cells. Much like an antibody, these fragments may specifically target cells expressing this antigen. Targeted delivery of these cytotoxic agents to the tumor cells would result in cell death and suppression of tumor growth. An example of the ability of an extracellular fragment binding to and activating its intact receptor (by homophilic binding) has been demonstrated with the CD84 receptor (Martin et al., J. Immunol. 167:3668-3676 (2001), herein incorporated by reference in its entirety).

Extracellular fragments of the BCLP receptor may also be used to modulate immune cells expressing the protein. Extracellular domain fragments of the cell surface antigen may bind to and activate its own receptor on the cell surface, which may result in stimulating the release of cytokines (such as interferon gamma from NK cells, T cells, B cells or myeloid cells, for example) that may enhance or suppress the immune system. Additionally, binding of these fragments to cells bearing BCLP may result in the activation of these cells and also may stimulate proliferation. Some fragments may bind to the intact cell surface antigen of the invention and block activation signals and cytokine release by immune cells. These fragments would then have an immunosuppressive effect. Fragments that activate and stimulate the immune system may have anti-tumor properties. These fragments may stimulate an immunological response that can result in immune-mediated tumor cell killing. The same fragments may result in stimulating the immune system to mount an enhanced response to foreign invaders such as viruses and bacteria. Fragments that suppress the immune response may be useful in treating lymphoproliferative disorders, auto-immune diseases, graft-vs-host disease, and inflammatory diseases, such as emphysema.

### 4.7 OTHER BINDING PEPTIDES OR SMALL MOLECULES

Screening of organic compound or peptide libraries with recombinantly expressed BCLP protein of the invention may be useful for identification of therapeutic molecules that function to specifically bind to or even inhibit the activity of BCLP proteins. Synthetic and naturally occurring products can be screened in a number of ways deemed routine to those of skill in the art. Random peptide libraries are displayed on phage (phage display) or on bacteria, such as on E. coli. These random peptide display libraries can be used to screen for peptides which interact with a known target which can be a protein or a polypeptide, such as a ligand or receptor, a biological or synthetic macromolecule, or organic or inorganic substances. By way of example, diversity libraries, such as random or combinatorial peptide or nonpeptide libraries can be screened for molecules that specifically bind to BCLP polypeptides. Many libraries are known in the art that can be used, i.e. chemically synthesized libraries, recombinant (i.e. phage display libraries), and in vitro translation-based libraries. Techniques for creating and screening such random peptide display libraries are known in the art (Ladner et al., U.S. Pat. No. 5,223, 409; Ladner et al., U.S. Pat. No. 4,946,778; Ladner et al., U.S. Pat. No. 5,403,484; Ladner et al., U.S. Pat. No. 5,571,698, all of which are herein incorporated by reference in their entirety) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.), and Pharmacia KLB Biotechnology Inc. (Piscataway, N.J.). Random peptide display libraries can be screened using the BCLP sequences disclosed herein to identify proteins which bind to the BCLP of the invention.

Examples of chemically synthesized libraries are described in Fodor et al., Science 251:767-773 (1991); Houghten et al., Nature 354:84-86 (1991); Lam et al., Nature 354:82-84 (1991); Medynski, Bio/Technology 12:709-710 (1994); Gallop et al., J. Med. Chem. 37:1233-1251 (1994); Ohlmeyer et al., Proc. Natl. Acad. Sci. USA 90:10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614-1618 (1994); Salmon et al., Proc. Natl. Acad. Sci. USA 90:11708-11712 (1993); PCT Publication No. WO 93/20242; Brenner and Lerner, Proc. Natl. Acad. Sci. USA 89:5381-5383 (1992), all of which are herein incorporated by reference in their entirety.

Examples of phage display libraries are described in Scott and Smith, Science 249:386-390 (1990); Devlin et al., Science 249:404-406 (1990); Christian et al., J. Mol. Biol. 227:711-718 (1992); Lenstra, J. Immunol Meth. 152:149-157 (1992); Kay et al., Gene 128:59-65 (1993); PCT Publication No. WO 94/18318, all of which are herein incorporated by reference in their entirety.

In vitro translation-based libraries include but are not limited to those described in PCT Publication No. WO 91/05058, and Mattheakis et al., Proc. Natl. Acad. Sci. USA 91:9022-9026 (1994), both of which are herein incorporated by reference in their entirety.

By way of examples of nonpeptide libraries, a benzodiazepine library (see for example, Bunin et al., Proc. Natl. Acad. Sci. USA 91:4708-4712 (1994), herein incorporated by reference in its entirety) can be adapted for use. Peptoid libraries (Simon et al., Proc. Natl. Acad. Sci. USA 89:9367-9371 (1992), herein incorporated by reference in its entirety) can also be used. Another example of a library that can be used, in which the amide functionalities in peptides have been permethylated to generate a chemically transformed combinatorial library, is described by Ostresh et al. (Proc. Natl. Acad. Sci. USA 91:11138-11142 (1994), herein incorporated by reference in its entirety).

Screening the libraries can be accomplished by any of a variety of commonly known methods. See, for example, the following references which disclose screening of peptide libraries: Parmley and Smith, Adv. Exp. Med. Biol. 251:215-218 (1989); Scott and Smith, Science 249:386-390 (1990); Fowlkes et al., Biotechniques 13:422-427 (1992); Oldenburg et al., Proc. Natl. Acad. Sci. USA 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. USA 89:6988-6992 (1992); Ellington et al., Nature 355:850-852 (1992); Rebar and Pabo, Science 263:671-673 (1993); and PCT Publication No. WO 94/18318, all of which are herein incorporated by reference in their entirety.

In a specific embodiment, screening can be carried out by contacting the library members with a BCLP protein (or nucleic acid or derivative) immobilized on a solid phase and harvesting those library members that bind to the protein (or nucleic acid or derivative). Examples of such screening methods, termed “panning” techniques are described by way of example in Parmley and Smith, Gene 73:305-318 (1988); Fowlkes et al., Biotechniques 13:422-427 (1992); PCT Publication No. WO 94/18318, all of which are herein incorporated by reference in their entirety, and in references cited hereinabove.

In another embodiment, the two-hybrid system for selecting interacting protein in yeast (Fields and Song, Nature 340:245-246 (1989); Chien et al., Proc. Natl. Acad. Sci. USA 88:9578-9582 (1991), both of which are herein incorporated by reference in their entirety) can be used to identify molecules that specifically bind to a BCLP protein or derivative.

These “binding polypeptides” or small molecules which interact with BCLP polypeptides of the invention can be used for tagging or targeting cells; for isolating homolog polypeptides by affinity purification; they can be directly or indirectly conjugated to drugs, toxins, radionuclides and the like. These binding polypeptides or small molecules can also be used in analytical methods such as for screening expression libraries and neutralizing activity, i.e., for blocking interaction between ligand and receptor, or viral binding to a receptor. The binding polypeptides or small molecules can also be used for diagnostic assays for determining circulating levels of BCLP polypeptides of the invention; for detecting or quantitating soluble BCLP polypeptides as marker of underlying pathology or disease. These binding polypeptides or small molecules can also act as BCLP “antagonists” to block BCLP binding and signal transduction in vitro and in vivo. These anti-BCLP binding polypeptides or small molecules would be useful for inhibiting BCLP activity or protein binding.

Binding polypeptides can also be directly or indirectly conjugated to drugs, toxins, radionuclides, prodrug-activating enzymes and the like, and these conjugates used for in vivo diagnostic or therapeutic applications. Binding peptides can also be fused to other polypeptides, for example an immunoglobulin constant chain or portions thereof, to enhance their half-life, and can be made multivalent (through, e.g. branched or repeating units) to increase binding affinity for the BCLP. For instance, binding polypeptides of the present invention can be used to identify or treat tissues or organs that express a corresponding anti-complementary molecule (receptor or antigen, respectively, for instance). More specifically, binding polypeptides or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the binding polypeptide, and include radionuclides, enzymes, substrates, cofactors, inhibitors, fluorescent markers, chemiluminescent markers, magnetic particles and the like. Suitable cytotoxic molecules may be directly or indirectly attached to the binding polypeptide, and include bacterial or plant toxins (for instance, diphtheria toxin, Pseudomonas exotoxin, ricin, abrin and the like), as well as therapeutic radionuclides, such as iodine-131, rhenium-188, or yttrium-90 (either directly attached to the binding polypeptide, or indirectly attached through a means of a chelating moiety, for instance). Binding polypeptides may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the binding polypeptide. For these purposes, biotin/streptavidin is an exemplary complementary/anticomplementary pair.

In another embodiment, binding polypeptide-toxin fusion proteins can be used for targeted cell or tissue inhibition or ablation (for instance, to treat cancer cells or tissues). Alternatively, if the binding polypeptide has multiple functional domains (i.e., an activation domain or a ligand binding domain, plus a targeting domain), a fusion protein including only the targeting domain may be suitable for directing a detectable molecule, a cytotoxic molecule, or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

### 4.8 DISEASES AMENABLE TO ANTI-BCLP TARGETING THERAPY

In one aspect, the present invention provides reagents and methods useful for treating diseases and conditions wherein cells that are associated with the disease or disorder express BCLP polypeptides. These diseases include cancers of the colon, breast, lung, ovary, pancreas, porstate, skin, and other solid tumors and hematopoietic-based cancers, and can include other hyperproliferative conditions, such as X-linked lymphoproliferative disorders, Epstein-Barr virus-related conditions such as mononucleosis, hyperplasia, psoriasis, contact dermatitis, and immunological disorders, arthritis, autoimmune disease, allergy, and inflammation. Whether the cells associated with a disease or condition express BCLP polypeptides can be determined using the diagnostic methods described herein.

Comparisons of BCLP mRNA and protein expression levels between diseased cells, tissue or fluid (blood, lymphatic fluid, etc.) and corresponding normal samples are made to determine if the patient will be responsive to BCLP therapy targeting BCLP antigens of the invention. Methods for detecting and quantifying the expression of BCLP polypeptide mRNA or protein use standard nucleic acid and protein detection and quantitation techniques that are well known in the art and are described in Sambrook, et al., Molecular Cloning: A Laboratory Manual, Cold Spring Harbor Laboratory, New York (1989) or Ausubel, et al., Current Protocols in Molecular Biology, John Wiley & Sons, New York, N.Y. (1989), both of which are incorporated herein by reference in their entirety. Standard methods for the detection and quantification of BCLP mRNA include in situ hybridization using labeled BCLP riboprobes (Gemou-Engesaeth, et al., Pediatrics 109: E24-E32 (2002), herein incorporated by reference in its entirety), Northern blot and related techniques using BCLP polynucleotide probes (Kunzli, et al., Cancer 94: 228 (2002), herein incorporated by reference in its entirety, herein incorporated by reference in its entirety), RT-PCR analysis using BCLP-specific primers (Angchaiskisiri, et al., Blood 99:130 (2002)), and other amplification detection methods, such as branched chain DNA solution hybridization assay (Jardi, et al., J. Viral Hepat. 8:465-471 (2001), herein incorporated by reference in its entirety), transcription-mediated amplification (Kimura, et al., J. Clin. Microbiol. 40:439-445 (2002)), microarray products, such as oligos, cDNAs, and monoclonal antibodies, and real-time PCR (Simpson, et al., Molec. Vision, 6:178-183 (2000), herein incorporated by reference in its entirety). Standard methods for the detection and quantification of BCLP protein include western blot analysis (Sambrook, et al., Molecular Cloning: A Laboratory Manual, Cold Spring Harbor Laboratory, New York (1989), Ausubel, et al., Current Protocols in Molecular Biology, John Wiley & Sons, New York, N.Y. (1989)), immunocytochemistry (Racila, et al., Proc. Natl. Acad. Sci. USA 95:4589-4594 (1998)supra), and a variety of immunoassays, including enzyme-linked immunosorbant assay (ELISA), radioimmuno assay (RIA), and specific enzyme immunoassay (EIA) (Sambrook, et al., Molecular Cloning: A Laboratory Manual, Cold Spring Harbor Laboratory, New York (1989), Ausubel, et al., Current Protocols in Molecular Biology, John Wiley & Sons, New York, N.Y. (1989)). Peripheral blood cells can also be analyzed for BCLP polypeptide expression using flow cytometry using, for example, immunomagnetic beads specific for BCLP polypeptides (Racila, et al., Proc. Natl. Acad. Sci. USA 95:4589-4594 (1998)) or biotinylated BCLP polypeptides antibodies (Soltys, et al., J. Immunol. 168:1903 (2002)). Tumor aggressiveness can be gauged by determining the levels of BCLP polypeptide or mRNA in tumor cells compared to the corresponding normal cells (Orlandi, et al., Cancer Res. 62:567 (2002)). In one embodiment, the disease or disorder is a cancer.

The cancers treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Tumors or neoplasms include growths of tissue cells in which the multiplication of the cells is uncontrolled and progressive. Some such growths are benign, but others are termed “malignant” and may lead to death of the organism. Malignant neoplasms or “cancers” are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and greater loss of their organization relative to one another and their surrounding tissues. This property is also called “anaplasia.”

Neoplasms treatable by the present invention also include solid phase tumors/malignancies, i.e., carcinomas, locally advanced tumors and human soft tissue sarcomas. Carcinomas include those malignant neoplasms derived from epithelial cells that infiltrate (invade) the surrounding tissues and give rise to metastastic cancers, including lymphatic metastases. Adenocarcinomas are carcinomas derived from glandular tissue, or which form recognizable glandular structures. Another broad category or cancers includes sarcomas, which are tumors whose cells are embedded in a fibrillar or homogeneous substance like embryonic connective tissue. The invention also enables treatment of cancers of the myeloid or lymphoid systems, including leukemias, lymphomas and other cancers that typically do not present as a tumor mass, but are distributed in the vascular or lymphoreticular systems.

The type of cancer or tumor cells that may be amenable to treatment according to the invention include hematopoietic-based cancers, for example, acute lymphocytic leukemia, acute nonlymphocytic leukemia, chronic lymphocytic leukemia, chronic myelocytic leukemia, cutaneous T-cell lymphoma, hairy cell leukemia, acute myeloid leukemia, erythroleukemia, chronic myeloid (granulocytic) leukemia, Hodgkin's disease, and non-Hodgkin's lymphoma.

The examples demonstrate that BCLP is expressed at high levels in colon tumors obtained from patients suffering from colon cancer, while BCLP is either absent or is expressed at low levels in healthy organs. Thus, colon cancer may be treated using the targeting compositions of the present invention.

Other solid tumors that may be targeted according to the invention include gastrointestinal cancers including esophageal cancer, stomach cancer, pancreatic cancer and gallbladder cancer, cancer of the adrenal cortex, ACTH-producing tumor, brain cancer including intrinsic brain tumors, neuroblastomas, astrocytic brain tumors, gliomas, and metastatic tumor cell invasion of the central nervous system, Ewing's sarcoma, head and neck cancer including mouth cancer and larynx cancer, kidney cancer including renal cell carcinoma, liver cancer, lung cancer including small and non-small cell lung cancers, malignant peritoneal effusion, malignant pleural effusion, skin cancers including malignant melanoma, tumor progression of human skin keratinocytes, squamous cell carcinoma, basal cell carcinoma, and hemangiopericytoma, mesothelioma, and Kaposi's sarcoma; bone cancer including osteomas and sarcomas such as fibrosarcoma and osteosarcoma; cancers of the female reproductive tract including uterine cancer, endometrial cancer, ovarian cancer, ovarian (germ cell) cancer and solid tumors in the ovarian follicle, vaginal cancer, cancer of the vulva, and cervical cancer; breast cancer (small cell and ductal); urologic cancers including penile cancer, testicular cancer, prostate cancer, and baldder cancer, and other cancers including retinoblastoma, thyroid cancer, trophoblastic neoplasms, and Wilms' tumor.

The invention is particularly illustrated herein in reference to treatment of certain types of experimentally defined cancers. In these illustrative treatments, standard state-of-the-art in vitro and in vivo models have been used. These methods can be used to identify agents that can be expected to be efficacious in in vivo treatment regimens. However, it will be understood that the method of the invention is not limited to the treatment of these tumor types, but extends to any cancer derived from any organ system. Leukemias can result from uncontrolled B cell proliferation initially within the bone marrow before disseminating to the peripheral blood, spleen, lymph nodes and finally to other tissues. Uncontrolled B cell proliferation also may result in the development of lymphomas that arise within the lymph nodes and then spread to the blood and bone marrow. Targeting BCLP polypeptides may be useful in treating B cell malignancies, leukemias, lymphomas and myelomas including but not limited to multiple myeloma, Burkitt's lymphoma, cutaneous B cell lymphoma, primary follicular cutaneous B cell lymphoma, B lineage acute lymphoblastic leukemia (ALL), B cell non-Hodgkin's lymphoma (NHL), B cell chronic lymphocytic leukemia (CLL), acute lymphoblastic leukemia, hairy cell leukemia (HCL), splenic marginal zone lymphoma, diffuse large B cell lymphoma, prolymphocytic leukemia (PLL), lymphoplasma cytoid lymphoma, mantle cell lymphoma, mucosa-associated lymphoid tissue (MALT) lymphoma, primary thyroid lymphoma, intravascular malignant lymphomatosis, splenic lymphoma, Hodgkin's Disease, intragraft angiotropic large-cell lymphoma, acute myelogenous leukemia, acute myelomonocytic leukemia, acute lymphoblastic leukemia, chronic myelogenic leukemia, malignant lymphoma, and lymphosarcoma cell leukemia. Other diseases that may be treated by the methods of the present invention include multicentric Castleman's disease, primary amyloidosis, Franklin's disease, Seligmann's disease, primary effusion lymphoma, post-transplant lymphoproliferative disease (PTLD) [associated with EBV infection.], paraneoplastic pemphigus, chronic lymphoproliferative disorders, X-linked lymphoproliferative syndrome (XLP), acquired angioedema, angioimmunoblastic lymphadenopathy with dysproteinemia, Herman's syndrome, post-splenectomy syndrome, congenital dyserythropoietic anemia type III, lymphoma-associated hemophagocytic syndrome (LAHS), necrotizing ulcerative stomatitis, Kikuchi's disease, lymphomatoid granulomatosis, Richter's syndrome, polycythemic vera (PV), Gaucher's disease, Gougerot-Sjogren syndrome, Kaposi's sarcoma, cerebral lymphoplasmocytic proliferation (Bind and Neel syndrome), X-linked lymphoproliferative disorders, pathogen associated disorders such as mononucleosis (Epstein Barr Virus), lymphoplasma cellular disorders, post-transplantational plasma cell dyscrasias, and Good's syndrome.

Autoimmune diseases, which can be associated with hyperactive B and T cell activity that results in autoantibody production. Additionally, autoimmune diseases can be associated with uncontrolled protease activity (Wernike et al., Arthritis Rheum. 46:64-74 (2002)) and aberrant cytokine activity (Rodenburg et al., Ann. Rheum. Dis. 58:648-652 (1999), both of which are herein incorporated by reference in their entirety). Inhibition of the development of autoantibody-producing cells or proliferation of such cells may be therapeutically effective in decreasing the levels of autoantibodies in autoimmune diseases. Inhibition of protease activity may reduce the extent of tissue invasion and inflammation associated with autoimmune diseases including but not limited to systemic lupus erythematosus, Hashimoto thyroiditis, Sjogren's syndrome, pericarditis luspus, Crohn's Disease, graft-verses-host disease, Graves' disease, myasthenia gravis, autoimmune hemolytic anemia, autoimmune thrombocytopenia, asthma, cryoglubulinemia, primary biliary sclerosis, pernicious anemia, Waldenstrom macroglobulinemia, hyperviscosity syndrome, macroglobulinemia, cold agglutinin disease, monoclonal gammopathy of undetermined origin, anetoderma and POEMS syndrome (polyneuropathy, organomegaly, endocrinopathy, M component, skin changes), connective tissue disease, multiple sclerosis, cystic fibrosis, rheumatoid arthritis, autoimmune pulmonary inflammation, psoriasis, Guillain-Barre syndrome, autoimmune thyroiditis, insulin dependent diabetes mellitis, autoimmune inflammatory eye disease, Goodpasture's disease, Rasmussen's encephalitis, dermatitis herpetiformis, thyoma, autoimmune polyglandular syndrome type 1, primary and secondary membranous nephropathy, cancer-associated retinopathy, autoimmune hepatitis type 1, mixed cryoglobulinemia with renal involvement, cystoid macular edema, endometriosis, IgM polyneuropathy (including Hyper IgM syndrome), demyelinating diseases including multiple sclerosis, angiomatosis, and monoclonal gammopathy.

Targeting BCLP polypeptides may also be useful in the treatment of allergic reactions and conditions e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis, allergic gastroenteropathy, inflammatory bowel disorder (IBD), and contact allergies, such as asthma (particularly allergic asthma), or other respiratory problems.

Targeting BCLP may also be useful in the management or prevention of transplant rejection in patients in need of transplants such as stem cells, tissue or organ transplant. Thus, one aspect of the invention may find therapeutic utility in various diseases (such as those usually treated with transplantation, including without limitation, aplastic anemia and paroxysmal nocturnal hemoglobinuria) as wells in repopulating the stem cell compartment post irridiation/chemotherapy, either in-vivo or ex-vivo (i.e. in conjunction with bone marrow transplantation or with peripheral progenitor cell transplantation (homologous or heterologous) as normal cells or genetically manipulated for gene therapy.

Targeting BCLP may also be possible to modulate immune responses, in a number of ways. Down regulation may be in the form of inhibiting or blocking an immune response already in progress or may involve preventing the induction of an immune response. Down regulating or preventing one or more antigen functions (including without limitation B lymphocyte antigen functions), e.g., modulating or preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a therapeutic composition of the invention may prevent cytokine synthesis by immune cells, such as T cells, and thus acts as an immunosuppressant. Moreover, a lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular therapeutic compositions in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA41g fusion proteins in vivo as described in Lenschow et al., Science 257:789-792 (1992) and Turka et al., Proc. Natl. Acad. Sci USA, 89:11102-11105 (1992), herein incorporated by reference in their entirety. In addition, murine models of GVHD (see Paul ed., Fundamental Immunology, Raven Press, New York, 1989, pp. 846-847, herein incorporated by reference in its entirety) can be used to determine the effect of therapeutic compositions of the invention on the development of that disease.

### 4.9 ADMINISTRATION

The BCLP targeting compositions used in the practice of a method of the invention may be formulated into pharmaceutical compositions comprising a carrier suitable for the desired delivery method. Suitable carriers include any material which when combined with the BCLP targeting compositions retain the anti-tumor function of the antibody and is nonreactive with the subject's immune systems. Examples include, but are not limited to, any of a number of standard pharmaceutical carriers such as sterile phosphate buffered saline solutions, bacteriostatic water, and the like.

The BCLP targeting compositions may be administered via any route capable of delivering the antibodies to the tumor site. Potentially effective routes of administration include, but are not limited to, intravenous, intraperitoneal, intramuscular, intratumor, intradermal, and the like. The preferred route of administration is by intravenous injection. A preferred formulation for intravenous injection comprises BCLP targeting compositions in a solution of preserved bacteriostatic water, sterile unpreserved water, and/or diluted in polyvinylchloride or polyethylene bags containing 0.9% sterile sodium chloride for Injection, USP. The BCLP targeting compositions may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the BCLP targeting composition via an acceptable route of administration such as intravenous injection (IV), typically at a dose in the range of about 0.1 to about 10 mg/kg body weight; however other exemplary doses in the range of 0.01 mg/kg to about 100 mg/kg are also contemplated. Doses in the range of 10-500 mg mAb per week may be effective and well tolerated. Rituximab (Rituxan®), a chimeric CD20 antibody used to treat B-cell lymphoma, non-Hodgkin's lymphoma, and relapsed indolent lymphoma, is typically administered at 375 mg/m² by IV infusion once a week for 4 to 8 doses. Sometimes a second course is necessary, but no more than 2 courses are allowed. An effective dosage range for Rituxan® would be 50 to 500 mg/m² (Maloney, et al., Blood 84: 2457-2466 (1994); Davis, et al., J. Clin. Oncol. 18: 3135-3143 (2000), both of which are herein incorporated by reference in their entirety). Based on clinical experience with Trastuzumab (Herceptin®), a humanized monoclonal antibody used to treat HER2(human epidermal growth factor 2)-positive metastatic breast cancer (Slamon, et al., Mol Cell Biol. 9: 1165 (1989), herein incorporated by reference in its entirety), an initial loading dose of approximately 4 mg/kg patient body weight IV followed by weekly doses of about 2 mg/kg IV of the BCLP targeting composition may represent an acceptable dosing regimen (Slamon, et al., N. Engl. J. Med. 344: 783(2001), herein incorporated by reference in its entirety). Preferably, the initial loading dose is administered as a 90 minute or longer infusion. The periodic maintenance dose may be administered as a 30 minute or longer infusion, provided the initial dose was well tolerated. However, as one of skill in the art will understand, various factors will influence the ideal dose regimen in a particular case. Such factors may include, for example, the binding affinity and half life of the mAb or mAbs used, the degree of BCLP overexpression in the patient, the extent of circulating shed BCLP antigen, the desired steady-state antibody concentration level, frequency of treatment, and the influence of chemotherapeutic agents used in combination with the treatment method of the invention.

Treatment can also involve BCLP targeting compositions conjugated to radioisotopes. Studies using radiolabeled-anticarcinoembryonic antigen (anti-CEA) monoclonal antibodies, provide a dosage guideline for tumor regression of 2-3 infusions of 30-80 mCi/m² (Behr, et al. Clin, Cancer Res. 5(10 Suppl.): 3232s-3242s (1999), Juweid, et al., J. Nucl. Med. 39:34-42 (1998), both of which are herein incorporated in their entirety).

Alternatively, dendritic cells transfected with mRNA encoding BCLP can be used as a vaccine to stimulate T-cell mediated anti-tumor responses. Studies with dendritic cells transfected with prostate-specific antigen mRNA suggest a 3 cycles of intravenous administration of 1×10⁷-5×10⁷ cells for 2-6 weeks concomitant with an intradermal injection of 10⁷ cells may provide a suitable dosage regimen (Heiser, et al., J. Clin. Invest. 109:409-417 (2002); Hadzantonis and O'Neill, Cancer Biother. Radiopharm. 1:11-22 (1999), both of which are herein incorporated in their entirety). Other exemplary doses of between 1×10⁵ to 1×10⁹ or 1×10⁶ to 1×10⁸ cells are also contemplated.

Naked DNA vaccines using plasmids encoding BCLP can induce an immunologic anti-tumor response. Administration of naked DNA by direct injection into the skin and muscle is not associated with limitations encountered using viral vectors, such as the development of adverse immune reactions and risk of insertional mutagenesis (Hengge, et al., J. Invest. Dermatol. 116:979 (2001), herein incorporated in its entirety). Studies have shown that direct injection of exogenous cDNA into muscle tissue results in a strong immune response and protective immunity (Ilan, Curr. Opin. Mol. Ther. 1:116-120 (1999), herein incorporated in its entirety). Physical (gene gun, electroporation) and chemical (cationic lipid or polymer) approaches have been developed to enhance efficiency and target cell specificity of gene transfer by plasmid DNA (Nishikawa and Huang, Hum. Gene Ther. 12:861-870 (2001), herein incorporated in its entirety). Plasmid DNA can also be administered to the lungs by aerosol delivery (Densmore, et al., Mol. Ther. 1:180-188 (2000)). Gene therapy by direct injection of naked or lipid—coated plasmid DNA is envisioned for the prevention, treatment, and cure of diseases such as cancer, acquired immunodeficiency syndrome, cystic fibrosis, cerebrovascular disease, and hypertension (Prazeres, et al., Trends Biotechnol. 17:169-174 (1999); Weihl, et al., Neurosurgery 44:239-252 (1999), both of which are herein incorporated in their entirety). HIV-1 DNA vaccine dose-escalating studies indicate administration of 30-300 pg/dose as a suitable therapy (Weber, et al., Eur. J. Clin. Microbiol. Infect. Dis. 20: 800 (2001), herin incorporated in its entirety. Naked DNA injected intracerebrally into the mouse brain was shown to provide expression of a reporter protein, wherein expression was dose-dependent and maximal for 150 μg DNA injected (Schwartz, et al., Gene Ther. 3:405-411 (1996), herein incorporated in its entirety). Gene expression in mice after intramuscular injection of nanospheres containing 1 microgram of beta-galactosidase plasmid was greater and more prolonged than was observed after an injection with an equal amount of naked DNA or DNA complexed with Lipofectamine (Truong, et al., Hum. Gene Ther. 9:1709-1717 (1998), herein incorporated in its entirety). In a study of plasmid-mediated gene transfer into skeletal muscle as a means of providing a therapeutic source of insulin, wherein four plasmid constructs comprising a mouse furin cDNA transgene and rat proinsulin cDNA were injected into the calf muscles of male Balb/c mice, the optimal dose for most constructs was 100 micrograms plasmid DNA (Kon, et al. J. Gene Med. 1:186-194 (1999), herein incorporated in its entirety). Other exemplary doses of 1 -1000 pg/dose or 10-500 pg/dose are also contemplated.

Optimally, patients should be evaluated for the level of circulating shed BCLP antigen in serum in order to assist in the determination of the most effective dosing regimen and related factors. Such evaluations may also be used for monitoring purposes throughout therapy, and may be useful to gauge therapeutic success in combination with evaluating other parameters.

### 4.9.1 BCLP Targeting Compositions

Compositions for targeting BCLP-expressing cells are within the scope of the present invention. Pharmaceutical compositions comprising antibodies are described in detail in, for example, U.S. Pat. No. 6,171,586, herein incorporated in its entirety. Such compositions comprise a therapeutically or prophylactically effective amount an antibody, or a fragment, variant, derivative or fusion thereof as described herein, in admixture with a pharmaceutically acceptable agent. Typically, the BCLP immunotargeting agent will be sufficiently purified for administration to an animal.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetraacetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18th Edition, Ed. A. R. Gennaro, Mack Publishing Company, (1990), herein incorporated in its entirety).

The optimal pharmaceutical composition will be determined by one skilled in the art depending upon, for example, the intended route of administration, delivery format, and desired dosage. See, for example, Remington's Pharmaceutical Sciences, supra. Such compositions may influence the physical state, stability, rate of in vivo release, and rate of in vivo clearance of the BCLP targeting agent.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present invention, BCLP immunotargeting agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The pharmaceutical compositions can be selected for parenteral delivery. Alternatively, the compositions may be selected for inhalation or for delivery through the digestive tract, such as orally. The preparation of such pharmaceutically acceptable compositions is within the skill of the art. The formulation components are present in concentrations that are acceptable to the site of administration. For example, buffers are used to maintain the composition at physiological pH or at slightly lower pH, typically within a pH range of from about 5 to about 8. When parenteral administration is contemplated, the therapeutic compositions for use in this invention may be in the form of a pyrogen-free, parenterally acceptable aqueous solution comprising the BCLP immunotargeting agent in a pharmaceutically acceptable vehicle. A particularly suitable vehicle for parenteral injection is sterile distilled water in which a BCLP immunotargeting agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

In another embodiment, a pharmaceutical composition may be formulated for inhalation. For example, a BCLP immunotargeting agent may be formulated as a dry powder for inhalation. Polypeptide or nucleic acid molecule inhalation solutions may also be formulated with a propellant for aerosol delivery. In yet another embodiment, solutions may be nebulized. Pulmonary administration is further described in PCT Application No. PCT/US94/001875, herein incorporated in its entirety, which describes pulmonary delivery of chemically modified proteins.

It is also contemplated that certain formulations may be administered orally. In one embodiment of the present invention, BCLP targeting agents that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the binding agent molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the BCLP immunotargeting agent may be dissolved or suspended in suitable liquids, such as fatty oils, liquid, or liquid polyethylene glycol with or without stabilizers.

Another pharmaceutical composition may involve an effective quantity of BCLP immunotargeting agent in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving BCLP immunotargeting agents in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT/US93/00829, herein incorporated in its entirety, that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919; European Patent No. EP 58,481), copolymers of L-glutamic acid and gamma ethyl-L-glutamate (Sidman et al., Biopolymers, 22:547-556 (1983)), poly (2-hydroxyethyl-methacrylate) (Langer et al., J Biomed Mater Res, 15:167-277, (1981)) and (Langer et al., Chem Tech, 12:98-105(1982)), ethylene vinyl acetate (Langer et al., supra) or poly-D (−)-3-hydroxybutyric acid (European Pat. No. EP 133,988, all of which are herein incorporated in their entirety). Sustained-release compositions also include liposomes, which can be prepared by any of several methods known in the art. See e.g., Epstein, et al., Proc Natl Acad Sci (USA), 82:3688-3692 (1985); European Pat. Nos. EP 36,676, EP 88,046, and EP 143,949, all of which are herein incorporated by reference in their entirety.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried BCLP immunotargeting agent and a second container having an aqueous formulation. Also included within the scope of this invention are kits containing single and multi-chambered pre-filled syringes (e.g., liquid syringes and lyosyringes).

### 4.9.2 Dosage

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which BCLP targeting agent is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 0.01 mg/kg to 1 g/kg; or 1 mg/kg up to about 100 mg/kg or 5 mg/kg up to about 100 mg/kg. In other embodiments, the dosage may range from 10 mCi to 100 mCi per dose for radioimmunotherapy, from about 1×10⁷-5×10⁷ cells or 1×10⁵ to 1×10⁹ cells or 1×10⁶ to 1×10⁸ cells per injection or infusion, or from 30 μg to 300 μg naked DNA per dose or 1-1000 μg/dose or 10-500 μg/dose, depending on the factors listed above.

For any compound, the therapeutically effective dose can be estimated initially either in cell culture assays or in animal models such as mice, rats, rabbits, dogs, or pigs. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the BCLP targeting agent in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

### 4.9.3 Routes of Administration

The route of administration of the pharmaceutical composition is in accord with known methods, e.g. orally, through injection by intravenous, intraperitoneal, intracerebral (intra-parenchymal), intracerebroventricular, intramuscular, intraocular, intra-arterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems, by implantation devices, or through inhalation. Where desired, the compositions may be administered by bolus injection or continuously by infusion, or by implantation device.

Alternatively or additionally, the composition may be administered locally via implantation of a membrane, sponge, or another appropriate material on to which the BCLP targeting agent has been absorbed or encapsulated. Where an implantation device is used, the device may be implanted into any suitable tissue or organ, and delivery of the BCLP targeting agent may be via diffusion, timed-release bolus, or continuous administration.

In some cases, it may be desirable to use pharmaceutical compositions in an ex vivo manner. In such instances, cells, tissues, or organs that have been removed from the patient are exposed to the pharmaceutical compositions after which the cells, tissues and/or organs are subsequently implanted back into the patient.

In other cases, a BCLP targeting agent can be delivered by implanting certain cells that have been genetically engineered to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

### 4.10 COMBINATION THERAPY

BCLP targeting agents of the invention can be utilized in combination with other therapeutic agents, and may enhance the effect of these other therapeutic agents such that a lesser daily amount, lesser total amount or reduced frequency of administration is required in order to achieve the same therapeutic effect at reduced toxicity. For cancer, these other therapeutics include, for example radiation treatment, chemotherapeutic agents, as well as other growth factors. For transplant rejection or autoimmune diseases, these other therapeutics include for example immunosuppressants such as cyclosporine, azathioprine corticosteroids, tacrolimus or mycophenolate mofetil.

In one embodiment, a BCLP targeting composition comprising an anti-BCLP antibody is used as a radiosensitizer. In such embodiments, the anti-BCLP antibody is conjugated to a radiosensitizing agent. The term “radiosensitizer,” as used herein, is defined as a molecule, preferably a low molecular weight molecule, administered to animals in therapeutically effective amounts to increase the sensitivity of the cells to be radiosensitized to electromagnetic radiation and/or to promote the treatment of diseases that are treatable with electromagnetic radiation. Diseases that are treatable with electromagnetic radiation include neoplastic diseases, benign and malignant tumors, and cancerous cells.

The terms “electromagnetic radiation” and “radiation” as used herein include, but are not limited to, radiation having the wavelength of 10-20 to 100 meters. Preferred embodiments of the present invention employ the electromagnetic radiation of: gamma-radiation (10⁻²⁰ to 10⁻¹³ m), X-ray radiation (10⁻¹² to 10⁻⁹ m), ultraviolet light (10 nm to 400 nm), visible light (400 nm to 700 nm), infrared radiation (700 nm to 1.0 mm), and microwave radiation (1 mm to 30 cm).

Radiosensitizers are known to increase the sensitivity of cancerous cells to the toxic effects of electromagnetic radiation. Many cancer treatment protocols currently employ radiosensitizers activated by the electromagnetic radiation of X-rays. Examples of X-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (lUdR), bromodeoxycytidine, fluorodeoxyuridine (FUdR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin(r), benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Chemotherapy treatment can employ anti-neoplastic agents including, for example, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2 ′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; ppipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as cisplatin and carboplatin, anthracenediones such as mitoxantrone, substituted urea such as hydroxyurea, methylhydrazine derivatives including N-methylhydrazine (MI H) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Combination therapy with growth factors can include cytokines, lymphokines, growth factors, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF0, TNF1, TNF2, G-CSF, Meg-CSF, GM-CSF, thrombopoietin, stem cell factor, and erythropoietin. Other compositions can include known angiopoietins, for example, vascular endothelial growth factor (VEGF). Growth factors include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-1 2, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil chemotactic factor 2, endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor 2, growth related protein, heparin binding epidermal growth factor, hepatocyte growth factor, hepatocyte growth factor receptor, insulin-like growth factor 1, insulin-like growth factor receptor, insulin-like growth factor II, insulin-like growth factor binding protein, keratinocyte growth factor, leukemia inhibitory factor, leukemia inhibitory factor receptor, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor, pre-B cell growth stimulating factor, stem cell factor, stem cell factor receptor, transforming growth factor, transforming growth factor 1, transforming growth factor 1.2, transforming growth factor 2, transforming growth factor 3, transforming growth factor 5, latent transforming growth factor 1, transforming growth factor binding protein I, transforming growth factor binding protein II, transforming growth factor binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

### 4.11 DIAGNOSTIC USES OF BCLP

### 4.11.1 Assays for Determining BCLP-Expression Status

Determining the status of BCLP expression patterns in an individual may be used to diagnose cancer and may provide prognostic information useful in defining appropriate therapeutic options. Similarly, the expression status of BCLP may provide information useful for predicting susceptibility to particular disease stages, progression, and/or tumor aggressiveness. The invention provides methods and assays for determining BCLP expression status and diagnosing cancers that express BCLP.

In one aspect, the invention provides assays useful in determining the presence of cancer in an individual, comprising detecting a significant increase or decrease, as applicable, in BCLP mRNA or protein expression in a test cell or tissue or fluid sample relative to expression levels in the corresponding normal cell or tissue. In one embodiment, the presence of BCLP mRNA is evaluated in tissue samples of a colon cancer. The presence of significant BCLP expression may be useful to indicate whether the colon cancer is susceptible to BCLP targeting using a targeting composition of the invention. In a related embodiment, BCLP expression status may be determined at the protein level rather than at the nucleic acid level. For example, such a method or assay would comprise determining the level of BCLP expressed by cells in a test tissue sample and comparing the level so determined to the level of BCLP expressed in a corresponding normal sample. In one embodiment, the presence of BCLP is evaluated, for example, using immunohistochemical methods. BCLP antibodies capable of detecting BCLP expression may be used in a variety of assay formats well known in the art for this purpose.

Peripheral blood may be conveniently assayed for the presence of cancer cells, including colon cancers, using RT-PCR to detect BCLP expression. The presence of RT-PCR amplifiable BCLP mRNA provides an indication of the presence of one of these types of cancer. A sensitive assay for detecting and characterizing cancer cells in blood may be used (Racila, et al., Proc. Natl. Acad. Sci. USA 95: 4589-4594 (1998), herein incorporated by reference in its entirety). This assay combines immunomagnetic enrichment with multiparameter flow cytometric and immunohistochemical analyses, and is highly sensitive for the detection of cancer cells in blood, reportedly capable of detecting one epithelial cell in 1 ml of peripheral blood.

A related aspect of the invention is directed to predicting susceptibility to developing cancer in an individual. In one embodiment, a method for predicting susceptibility to cancer comprises detecting BCLP mRNA or BCLP protein in a tissue sample, its presence indicating susceptibility to cancer, wherein the degree of BCLP mRNA expression present is proportional to the degree of susceptibility.

Yet another related aspect of the invention is directed to methods for assessment of tumor aggressiveness (Orlandi, et al., Cancer Res. 62:567 (2002), herein incorporated by reference in its entirety). In one embodiment, a method for gauging aggressiveness of a tumor comprises determining the level of BCLP mRNA or BCLP protein expressed by cells in a sample of the tumor, comparing the level so determined to the level of BCLP mRNA or BCLP protein expressed in a corresponding normal tissue taken from the same individual or a normal tissue reference sample, wherein the degree of BCLP mRNA or BCLP protein expression in the tumor sample relative to the normal sample indicates the degree of aggressiveness.

Methods for detecting and quantifying the expression of BCLP mRNA or protein are described herein and use standard nucleic acid and protein detection and quantification technologies well known in the art. Standard methods for the detection and quantification of BCLP mRNA include in situ hybridization using labeled BCLP riboprobes (Gemou-Engesaeth, et al., Pediatrics, 109:E24-E32 (2002)), Northern blot and related techniques using BCLP polynucleotide probes (Kunzli, et al., Cancer 94:228 (2002)), RT-PCR analysis using primers specific for BCLP (Angchaiskisiri, et al., Blood99:130 (2002)), and other amplification type detection methods, such as, for example, branched DNA (Jardi, et al., J. Viral Hepat. 8:465-471 (2001)), SISBA, TMA (Kimura, et al., J. Clin. Microbiol. 40:439-445 (2002)), and microarray products of a variety of sorts, such as oligos, cDNAs, and monoclonal antibodies. In a specific embodiment, real-time RT-PCR may be used to detect and quantify BCLP mRNA expression (Simpson, et al., Molec. Vision 6:178-183 (2000)). Standard methods for the detection and quantification of protein may be used for this purpose. In a specific embodiment, polyclonal or monoclonal antibodies specifically reactive with the wild-type BCLP may be used in an immunohistochemical assay of biopsied tissue (Ristimaki, et al., Cancer Res. 62:632 (2002), herein incorporated by reference in its entirety).

### 4.11.2 Medical Imaging

BCLP antibodies that recognize BCLP and fragments thereof are useful in medical imaging of sites expressing BCLP. Such methods involve chemical attachment of a labeling or imaging agent, such as a radioisotope, which include ⁶⁷Cu, ⁹⁰Y, ¹²⁵I, ¹³¹I, ¹⁸⁶Re, ¹⁸⁸Re, ²¹¹At, ²¹²Bi, administration of the labeled antibody and fragment to a subject in a pharmaceutically acceptable carrier, and imaging the labeled antibody and fragment in vivo at the target site. Radiolabelled anti-BCLP antibodies or fragments thereof may be particularly useful in in vivo imaging of BCLP expressing cancers, such as lymphomas or leukemias. Such antibodies may provide highly sensitive methods for detecting metastasis of BCLP-expressing cancers.

Upon consideration of the present disclosure, one of skill in the art will appreciate that many other embodiments and variations may be made in the scope of the present invention. Accordingly, it is intended that the broader aspects of the present invention not be limited to the disclosure of the following examples.

### 5. EXAMPLES

### Examples 1

### The mRNA Encoding BCLP is Highly Expressed in Colon Tumors

FIG. 4 shows the relative expression of BCLP mRNA that was derived from healthy tissues, and from colon tumors from patients.

Total mRNA derived from the colon tumors (HTB37, CCL233, HTB38, CO8067T, CO7932T, H03-130T, COLON T, CO7413T, H03-128T, H03-134T, H03-132T, H03-126T), tissue adjacent to the colon tumors (CO8067N,CO7932N, COLON N, H03-133N, H03-129N, H03-131 N, H03-135N, H03-127N), and the total mRNA derived from lung, kidney, small intestine, brain, colon, pancreas, adrenal gland, heart, skeletal muscle, liver, and was purchased from Clinomics Biosciences Inc., (Pittsfield, Mass.). The RNA was subjected to quantitative real-time PCR (TaqMan) (Simpson et al., Molec Vision 6:178-183 (2000)) to determine the relative expression of BCLP in human tissues. The forward and reverse primers that were used in the PCR reactions were: 5′ TGGCCCTCGCACCTGA 3′ (forward; SEQ ID NO: 20), and 5′ GGCACAGGCTGGAGCTATAAA 3′ (reverse; SEQ ID NO: 21), respectively. DNA sequences encoding Elongation Factor 1 were used as a positive control and normalization factors in all samples. All assays were performed in duplicate with the resulting values averaged.

The Y axis shows the relative fold expression of the mRNA as determined by the number of cycles required to amplify the signal from the mRNA. The larger the number of PCR cycles, the lower the amount of mRNA present in the tissue. The level of expression is reported as being relative to the lowest level detected in a sample that was set equal to 1. Absence of signal indicates complete absence of mRNA.

FIG. 4 shows that BCLP mRNA is expressed at high levels in colon tumors and in tissue adjacent to the colon tumors, relative to its expression in healthy organs including lung, kidney, small intestine, brain, colon, pancreas, adrenal gland, heart, skeletal muscle, liver, and spleen. The results indicate that BCLP polypeptide may be used as an immunotherapeutic antibody target or as a diagnostic marker for colon cancer.

### Example 2

### Production of BCLP-Specific Antibodies

Cells expressing BCLP were identified using antibodies to BCLP. Polyclonal antibodies were produced by injection of peptide antigens into rabbits. Rabbits were immunized with a peptide that was predicted to be immunogenic, and having amino acid sequence Gly Lys Ser Ser His His Met Met Arg Glu Asn Pro Glu Leu Val Glu Gly Arg Asp (SEQ ID NO: 22) that was conjugated to KLH (keyhole limpet hemocyanin). The rabbit was initially immunized with conjugated peptide in complete Freund's adjuvant, followed by a booster shot every two weeks with injections of conjugated peptide in incomplete Freund's adjuvant. Anti-BCLP antibody was affinity purified from rabbit serum using BCLP peptide coupled to Affi-Gel 10 (Bio-Rad), and stored in phosphate-buffered saline with 0.1% sodium azide. To determine that the polyclonal antibodies were BCLP-specific, an expression vector encoding BCLP were introduced into mammalian cells. Western blot analysis of protein extracts of non-transfected cells and the BCLP-containing cells was performed using the polyclonal antibody sample as the primary antibody and a horseradish peroxidase-labeled anti-rabbit antibody as the secondary antibody. Detection of a band corresponding to BCLP in the BCLP-containing cells and lack thereof in the control cells indicated that the polyclonal antibodies selectively bound BCLP (data not shown).

Monoclonal antibodies may also be produced by injecting mice with a BCLP peptide, with or without adjuvant. Subsequently, the mouse is boosted every 2 weeks until an appropriate immune response has been identified (typically 1-6 months), at which point the spleen is removed. The spleen is minced to release splenocytes, which are fused (in the presence of polyethylene glycol) with murine myeloma cells. The resulting cells (hybridomas) are grown in culture and selected for antibody production by clonal selection. The antibodies are secreted into the culture supernatant, facilitating the screening process, such as screening by an enzyme-linked immunosorbent assay (ELISA). Alternatively, humanized monoclonal antibodies are produced either by engineering a chimeric murine/human monoclonal antibody in which the murine-specific antibody regions are replaced by the human counterparts and produced in mammalian cells, or by using transgenic “knock out” mice in which the native antibody genes have been replaced by human antibody genes and immunizing the transgenic mice as described above.

### Example 3

### Methods Using BCLP-Specific Antibodies to Detect BCLP in Human Tissues

Expression of BCLP in human cancerous tissues was detected using the rabbit polyclonal anti-BCLP antibodies described in Example 2. The anti-BCLP polyclonal antibody was optimized for use in immunohistochemistry, and screened across panels containing human tissues. The specificity of binding was ascertained by the ability of the immunogenic peptide to block the binding of the anti-BCLP antibody. The specificity of binding was validated by incubating tissue sections with either 2.5 μg/ml or 5.0 μg/ml anti-BCLP antibody in the presence or absence of immunogenic peptide at molar ratios of 1:1, 1:10, and 1:100 antibody:peptide for 60 minutes at room temperature. Specific binding of the antibody to the BCLP target antigen was detected using the anti-rabbit IgG biotinylated secondary antibody and the reagents contained in the Vectastain© ABC-AP Kit AK-5001. The binding was visualized using the Vector© Red Alkaline Phosphatase Substrate Kit SK-5100 (©Vector Laboratories, Inc., Burlingame, Calif.) according to the methods provided by the manufacturer.

Immunohistochemistry studies were performed with BCLP-antibodies on a general human cancer screen multi-tissue array (LifeSpan Biosciences, Inc., Seatlle, Wash.). The array comprised human tissues that had been prepared for immunohistochemical analysis (IHC) by fixing tissues in 10%formalin, embedding in paraffin, and sectioned using standard techniques. Binding of anti-BCLP antibody to all tissue sections was performed using 2.5 pg/ml anti-BCLP antibody in the presence or absence of a ten-fold molar excess of immunogenic peptide. Sections were stained using the BCLP-specific antibody followed by incubation with a secondary horse radish peroxidase (HRP)-conjugated antibody and visualized by the product of the HRP enzymatic reaction. The intensity of the stain was scored 1-4; with scores of 3 and 4 reflecting the most intense staining, and the most significant expression of BCLP.

The cellular expression of BCLP is summarized in Table 1.

TABLE 1

| Malignant cells | Intensity |
|---|---|
| Breast carcinoma | |
| Female patient of unknown age | 3 (Occasional)-4 (Rare)0 |
| Male patient (82 years) | 3 (Many)-4 (Many) |
| Female patient (77 years) | 2 (Many)-4 (Many) |
| Colon carcinoma | |
| Male (78 years) | 2 (Most) |
| Male (77 years) | 1 (Many)-3 (Occasional) |
| Male (79 years) | 2 (Many)-3 (Many) |
| Non-small cell lung carcinoma | |
| Unknown age and sex | 2 (Many) |
| Unknown age and sex | 2 (Most)-3 (Rare) |
| Male (66 years) | 2 (Many)-4 (Occasional) |
| Small cell lung carcinoma | |
| Male (60 years) | 2 (Occasional)-4 (Occasional) |
| Female (53 years) | 2 (Many)-3 (Occasional) |
| Male (86 years) | 1-2 |
| Ovarian carcinoma | |
| Female (37 years) | 2 (Many) |
| Female (46 years) | 3 (Most)-4 (Occasional) |
| Female (71 years) | 1 (Many)-2 (Many) |
| Pancreatic carcinoma | |
| Male (55 years) | 1 (Many)-3 (Occasional) |
| Unknown sex (43 years) | 3 (Many)-4 (Occasional) |
| Female (41 years) | 2 (Many)-4 (Many) |
| Prostate carcinoma | |
| Male 62 ears | 1 (Many)-2 (Many) |
| Male 78 ears | 2 (Many)-4 (Occasional) |
| Male (85 years) | 1 (Most)-2 (Rare) |
| Skin Melanoma | |
| Male (75 years) | 1 (Many)-3 (Rare) |
| Male (46 years) | 1 (Most)-2 (Rare) |
| Male 31 ears | 2 Most-2 Rare |

Specific binding of BCLP antibodies was seen in tissue samples of carcinomas of the breast, colon, lung (non-small cell and small cell lung carcinoma), ovary, pancreas, prostate, and in melanoma.

These data indicate that BCLP may be used as a therapeutic target or as a diagnostic marker for these disorders.

### Example 4

### In Vitro Antibody-Dependent Cytotoxicity Assay

The ability of a BCLP-specific antibody to induce antibody-dependent cell-mediated cytoxicity (ADCC) is determined in vitro. ADCC is performed using the CytoTox 96 Non-Radioactive Cytoxicity Assay (Promega; Madison, Wis.) (Hornick et al., Blood 89:4437-4447, (1997)) as well as effector and target cells. Peripheral blood mononuclear cells (PBMC) or neutrophilic polymorphonuclear leukocytes (PMN) are two examples of effector cells that can be used in this assay. PBMC are isolated from healthy human donors by Ficoll-Paque gradient centrifugation, and PMN are purified by centrifugation through a discontinuous percoll gradient (70% and 62%) followed by hypotonic lysis to remove residual erythrocytes. Colon cancer cells (for example) are used as target cells.

Colon cancer cells are suspended in RPMI 1640 medium supplemented with 2% fetal bovine serum and plated in 96-well V-bottom microtitier plates at 2×10⁴ cells/well. BCLP-specific antibody is added in triplicate to individual wells at 1 □g/ml, and effector cells are added at various effector:target cell ratios (12.5:1 to 50:1). The plates are incubated for 4 hours at 37° C. The supernatants are then harvested, lactate dehydrogenase release determined, and percent specific lysis calculated using the manufacture's protocols.

### Example 5

### Toxin-Conjugated BCLP Specific Antibodies

Antibodies to BCLP are conjugated to toxins and the effect of such conjugates in animal models of cancer is evaluated. Chemotherapeutic agents, such as calicheamycin and carboplatin, or toxic peptides, such as ricin toxin, are used in this approach. Antibody-toxin conjugates are used to target cytotoxic agents specifically to cells bearing the antigen. The antibody-toxin binds to these antigen-bearing cells, becomes internalized by receptor-mediated endocytosis, and subsequently destroys the targeted cell. In this case, the antibody-toxin conjugate targets colon cancer BCLP-expressing cells, and delivers the cytotoxic agent to the tumor resulting in the death of the tumor cells.

One such example of a toxin that may be conjugated to an antibody is carboplatin. The mechanism by which this toxin is conjugated to antibodies is described in Ota et al., Asia-Oceania J. Obstet. Gynaecol. 19: 449-457 (1993). The cytotoxicity of carboplatin-conjugated BCLP-specific antibodies is evaluated in vitro, for example, by incubating BCLP-expressing target cells with various concentrations of conjugated antibody, medium alone, carboplatin alone, or antibody alone. The antibody-toxin conjugate specifically targets and kills cells bearing the BCLP antigen, whereas, cells not bearing the antigen, or cells treated with medium alone, carboplatin alone, or antibody alone, show no cytotoxicity.

The antitumor efficacy of carboplatin-conjugated BCLP-specific antibodies is demonstrated in in vivo murine tumor models. Five to six week old, athymic nude mice are engrafted with tumors subcutaneously or through intravenous injection. Mice are treated with the BCLP-carboplatin conjugate or with a non-specific antibody-carboplatin conjugate. Tumor xenografts in the mouse bearing the BCLP antigen are targeted and bound to by the BCLP-carboplatin conjugate. This results in tumor cell killing as evidenced by tumor necrosis, tumor shrinkage, and increased survival of the treated mice.

Other toxins are conjugated to BCLP-specific antibodies using methods known in the art. An example of a toxin conjugated antibody in human clinical trials is CMA-676, an antibody to the CD33 antigen in AML which is conjugated with calicheamicin toxin (Larson, Semin. Hematol. 38(Suppl 6):24-31 (2001)).

### Example 6

### Radio-Immunotherapy Using BCLP-Specific Antibodies

Animal models are used to assess the effect of antibodies specific to BCLP as vectors in the delivery of radionuclides in radio-immunotherapy to treat colon cancer, hematological malignancies, and solid tumors. Human tumors are propagated in 5-6 week old athymic nude mice by injecting a carcinoma cell line or tumor cells subcutaneously. Tumor-bearing animals are injected intravenously with radio-labeled anti-BCLP antibody (labeled with 30-40 μCi of ¹³¹I, for example) (Behr, et al., Int. J. Cancer 77: 787-795 (1988)). Tumor size is measured before injection and on a regular basis (i.e. weekly) after injection and compared to tumors in mice that have not received treatment. Anti-tumor efficacy is calculated by correlating the calculated mean tumor doses and the extent of induced growth retardation. To check tumor and organ histology, animals are sacrificed by cervical dislocation and autopsied. Organs are fixed in 10% formalin, embedded in paraffin, and thin sectioned. The sections are stained with hematoxylin-eosin.

### Example 7

### Therapy Using BCLP-Specific Antibodies

Animal models are used to evaluate the effect of BCLP-specific antibodies as targets for antibody-based immunotherapy using monoclonal antibodies. Human colon cancer cells are injected into the tail vein of 5-6 week old nude mice whose natural killer cells have been eradicated. To evaluate the ability of BCLP-specific antibodies in preventing tumor growth, mice receive an intraperitoneal injection with BCLP-specific antibodies either 1 or 15 days after tumor inoculation followed by either a daily dose of 20 μg or 100 μg once or twice a week, respectively (Ozaki, et al., Blood 90:3179-3186 (1997)). Levels of human IgG (from the immune reaction caused by the human tumor cells) are measured in the murine sera by ELISA.

The effect of BCLP-specific antibodies on the proliferation of colon cancer cells is examined in vitro using a ³H-thymidine incorporation assay (Ozaki et al., supra). Cells are cultured in 96-well plates at 1×10⁵ cells/ml in 100 μl/well and incubated with various amounts of BCLP antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

The cytotoxicity of the BCLP monoclonal antibody is examined by the effect of complements on colon cancer cells using a ⁵¹Cr-release assay (Ozaki et al., supra). Colon cancer cells are labeled with 0.1 mCi ⁵¹Cr-sodium chromate at 37° C. for 1 h. ⁵¹Cr-labeled cells are incubated with various concentrations of BCLP monoclonal antibody or control IgG on ice for 30 min. Unbound antibody is removed by washing with medium. Cells are distributed into 96-well plates and incubated with serial dilutions of baby rabbit complement at 37° C. for 2 h. The supernatants are harvested from each well and the amount of ⁵¹Cr released is measured using a gamma counter. Spontaneous release of ⁵¹Cr is measured by incubating cells with medium alone, whereas maximum ⁵¹Cr release is measured by treating cells with 1 % NP-40 to disrupt the plasma membrane. Percent cytotoxicity is measured by dividing the difference of experimental and spontaneous ⁵¹Cr release by the difference of maximum and spontaneous ⁵¹Cr release.

Antibody-dependent cell-mediated cytotoxicity (ADCC) for the BCLP monoclonal antibody is measured using a standard 4 h ⁵¹Cr-release assay (Ozaki et al., supra). Splenic mononuclear cells from SCID mice are used as effector cells and cultured with or without recombinant interleukin-2 (for example) for 6 days. 5° Cr-labeled target colon cancer cells (1×10⁴ cells) are placed in 96-well plates with various concentrations of anti-BCLP monoclonal antibody or control IgG. Effector cells are added to the wells at various effector to target ratios (12.5:1 to 50:1). After 4 h, culture supernatants are removed and counted in a gamma counter. The percentage of cell lysis is determined as above.

### Example 8

### BCLP-Specific Antibodies as Immunosuppressants

Animal models are used to assess the effect of BCLP-specific antibodies block signaling through the BCLP receptor to suppress autoimmune diseases, such as arthritis or other inflammatory conditions, or rejection of organ transplants. Immunosuppression is tested by injecting mice with horse red blood cells (HRBCs) and assaying for the levels of HRBC-specific antibodies (Yang, et al., Int. Immunopharm. 2:389-397 (2002)). Animals are divided into five groups, three of which are injected with anti-BCLP antibodies for 10 days, and 2 of which receive no treatment. Two of the experimental groups and one control group are injected with either Earle's balanced salt solution (EBSS) containing 5-10×10⁷ HRBCs or EBSS alone. Anti- BCLP antibody treatment is continued for one group while the other groups receive no antibody treatment. After 6 days, all animals are bled by retro-orbital puncture, followed by cervical dislocation and spleen removal. Splenocyte suspensions are prepared and the serum is removed by centrifugation for analysis.

Immunosupression is measured by the number of B cells producing HRBC-specific antibodies. The Ig isotype (for example, IgM, IgG1, IgG2, etc.) is determined using the IsoDetect™ Isotyping kit (Stratagene, La Jolla, Calif.). Once the Ig isotype is known, murine antibodies against HRBCs are measured using an ELISA procedure. 96-well plates are coated with HRBCs and incubated with the anti-HRBC antibody-containing sera isolated from the animals. The plates are incubated with alkaline phosphatase-labeled secondary antibodies and color development is measured on a microplate reader (SPECTRAmax 250, Molecular Devices) at 405 nm using p-nitrophenyl phosphate as a substrate.

Lymphocyte proliferation is measured in response to the T and B cell activators concanavalin A and lipopolysaccharide, respectively (Jiang, et al., J. Immunol. 154:3138-3146 (1995). Mice are randomly divided into 2 groups, 1 receiving anti-BCLP antibody therapy for 7 days and 1 as a control. At the end of the treatment, the animals are sacrificed by cervical dislocation, the spleens are removed, and splenocyte suspensions are prepared as above. For the ex vivo test, the same number of splenocytes are used, whereas for the in vivo test, the anti-BCLP antibody is added to the medium at the beginning of the experiment. Cell proliferation is also assayed using the ³H-thymidine incorporation assay described above (Ozaki, et al., Blood 90: 3179 (1997)).

### Example 9

### Cytokine Secretion in Response to BCLP Peptide Fragments

Assays are carried out to assess activity of fragments of the BCLP protein, such as the Ig domain, to stimulate cytokine secretion and to stimulate immune responses in NK cells, B cells, T cells, and myeloid cells. Such immune responses can be used to stimulate the immune system to recognize and/or mediate tumor cell killing or suppression of growth. Similarly, this immune stimulation can be used to target bacterial or viral infections. Alternatively, fragments of the BCLP that block activation through the BCLP receptor may be used to block immune stimulation in natural killer (NK), B, T, and myeloid cells.

Fusion proteins containing fragments of the BCLP, such as the Ig domain (BCLP-Ig), are made by inserting a CD33 leader peptide, followed by a BCLP domain fused to the Fc region of human IgG1 into a mammalian expression vector, which is stably transfected into NS-1 cells, for example. The fusion proteins are secreted into the culture supernatant, which is harvested for use in cytokine assays, such as interferon-γ (IFN-γ) secretion assays (Martin, et al., J. Immunol. 167:3668-3676 (2001)).

PBMCs are activated with a suboptimal concentration of soluble CD3 and various concentrations of purified, soluble anti-BCLP monoclonal antibody or control IgG. For BCLP-Ig cytokine assays, anti-human Fc Ig at 5 or 20 pg/ml is bound to 96-well plates and incubated overnight at 4° C. Excess antibody is removed and either BCLP-Ig or control Ig is added at 20-50 μg/ml and incubated for 4 h at room temperature. The plate is washed to remove excess fusion protein before adding cells and anti-CD3 to various concentrations. Supernatants are collected after 48 h of culture and IFN-γ levels are measured by sandwich ELISA, using primary and biotinylated secondary anti-human IFN-γ antibodies as recommended by the manufacturer.

### Example 10

### Diagnostic Methods Using BCLP-Specific Antibodies to Detect BCLP Expression

Expression of BCLP in tissue samples (normal or diseased) is detected using anti-BCLP antibodies. Samples are prepared for immunohistochemical (IHC) analysis by fixing the tissue in 10% formalin embedding in paraffin, and sectioning using standard techniques. Sections are stained using the BCLP-specific antibody followed by incubation with a secondary horse radish peroxidase (HRP)-conjugated antibody and visualized by the product of the HRP enzymatic reaction.

Expression of BCLP on the surface of cells within a blood sample is detected by flow cytometry. Peripheral blood cells are isolated from a blood sample using standard techniques. The cells are washed with ice-cold PBS and incubated on ice with the BCLP-specific polyclonal antibody for 30 min. The cells are gently pelleted, washed with PBS, and incubated with a fluorescent anti-rabbit antibody for 30 min. on ice. After the incubation, the cells are gently pelleted, washed with ice cold PBS, and resuspended in PBS containing 0.1% sodium azide and stored on ice until analysis. Samples are analyzed using a FACScalibur flow cytometer (Becton Dickinson) and CELLQuest software (Becton Dickinson). Instrument setting are determined using FACS-Brite calibration beads (Becton-Dickinson).

Tumors expressing BCLP are imaged using BCLP-specific antibodies conjugated to a radionuclide, such as ¹²³I, and injected into the patient for targeting to the tumor followed by X-ray or magnetic resonance imaging.

### Example 11

### Tumor Imaging Using BCLP-Specific Antibodies

BCLP-specific antibodies are used for imaging BCLP-expressing cells in vivo. Six-week-old athymic nude mice are irradiated with 400 rads from a cesium source. Three days later the irradiated mice are inoculated with 4×10⁷ RA1 cells and 4×10⁶ human fetal lung fibroblast feeder cells subcutaneously in the thigh. When the tumors reach approximately 1 cm in diameter, the mice are injected intravenously with an inoculum containing 100 μCi/10 μg of ¹³¹I-labeled BCLP-specific antibody. At 1, 3, and 5 days postinjection, the mice are anesthetized with a subcutaneous injection of 0.8 mg sodium pentobarbital. The immobilized mice are then imaged in a prone position with a Spectrum 91 camera equipped with a pinhole collimator (Raytheon Medical Systems; Melrose Park, Ill.) set to record 5,000 to 10,000 counts using the Nuclear MAX Plus image analysis software package (MEDX Inc.; Wood Dale, Ill.) (Hornick, et al., Blood 89:4437-4447 (1997)).

### Example 12

### In Vitro Tumor Suppression Assays

To determine the effect of a BCLP polypeptide of the invention on tumor growth, cells expressing BCLP polypeptides are produced by liposome-mediated transfection of the tumorgenic human prostate epithelial cell line, M12, using Tfx-50 according to the manufacture's protocol and using DNA in a 60-mm tissue culture dish. Transfecting the M12 cells with a mammalian expression vector alone produces control cells. Both transfected and controltransfected cells are maintained with G418 and the formation of individual colonies are monitored. Visible colonies are subcloned, using cloning rings, and each colony is transferred to a new well in a 12-well tissue culture plate. Cells are grown to confluence and split twice before the medium is collected, and total cytoplasmic RNA is isolated.

Western immunoblots are carried out by collecting media from the cells and normalizing based on the cell counts and concentrating by filtrating through nitrocellulose (Birnbaum et al., J. Endocrinology, 141:535-540 (1994), herein incorporated by reference in its entirety). After concentration, proteins are redissolved in a mixture of SDS sample buffer (0.5 M Tris (pH 6.8)), 1% SDS, 10% glycerol, 0.003% bromphenol blue, and 8M urea by heating for 10 minutes at 100° C. Samples are electrophoresed on 12% SDS-polyacrylamide gels and then electroblotted onto nitrocellulose. Western blots are incubated with BCLP antiserum at a 1:3000 dilution in 0.3% Tween 20 in Tris buffered saline (TBS) overnight at 4° C. Bound antibody is detected using a horseradish peroxidase-linked donkey antirabbit secondary antibody and the ECL detection system according to the manufacturer's protocol. Ligands blots were performed as described in the art (Damon et al., Endocrinology 139:3456-3464 (1998), herein incorporated by reference in its entirety).

Selected cell lines found to be expressing high levels of BCLP polypeptides would then be used in growth assays. Cell growth and proliferation would be monitored by cell counts over the course of 2 weeks. Suppression of tumor cell growth by BCLP polypeptides would be demonstrated by a reduction in cell number relative to the control cells over the course of the assay. Suppression of cell growth may be a result of a reduction in the rate of proliferation or by in increase in tumor cell apoptosis relative to control.

### Example 13

### In Vivo Tumor Models

The tumor suppressing activity of BCLP targeting molecules is tested by taking groups of 4-10 nude, athymic male mice are injected subcutaneously with 10⁶ cells, either a control (M12pcDNA), BCLP-expressing clones, or low expressing clones (Spenger et al., Cancer Research 59:2370-2375 (1999), incorporated herein by reference in its entirety). The clones that have the lowest levels of BCLP are used as the comparison benchmark. Mice are monitored for 8 weeks for weight gain/loss and tumor formation. Tumor volume is calculated using the formula (l×w²)/2 (where l=length and w=width of the tumor) (Id.).

Statistical analysis using the Kruskal-Wallis method for comparing tumor formation, and the Mann-Whitney U test for comparing tumor volume are performed to determine any statistical significance amongst groups.

After 8 weeks, the mice are sacrificed, and the tumors removed and digested with 0.1% collagenase (Type I) and 50 μg/ml DNase (Worthington Biochemical Corp., Freehold, N.J.). Dispersed cells are plated in ITS medium/5% FBS at %% CO₂ at 37° C. for 24 hours to allow attachment. After 24 hours, the cultures are switched to serum-free medium. The cells are split, the media and RNA collected, and Western immunoblots and Northern blots are done to detect BCLP.

### Example 14

### In Vitro Assay of Cell Proliferation and Migration

The effect of BCLP-specific antibodies or therapeutic peptides on the proliferation of colon cancer cells is examined in vitro using a ³H-thymidine incorporation assay (Ozaki et al., Blood 90:3179-3186 (1997), herein incorporated by reference in its entirety. Tumor cells are cultured in 96-well plates at 1×10⁵ cells/ml in 100 μl/well and incubated with various amounts of antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

Cell migration is conducted in 24-well, 6.5-mm internal diameter Transwell cluster plates (Corning Costar, Cambridge, Mass.). Briefly, 10⁵ cells/75 μl are loaded onto fibronectin (5 μM)-coated polycarbonate membranes (8-μm pore size) separating two chambers of a transwell (Tai et al., Blood 99:1419-1427 (2002), herein incorporated by reference in its entirety. Medium with or without anti-BCLP antibodies is added to the lower chamber of the Transwell cluster plates. After 8-16 h, cells migrating to the lower chamber are counted using a Coulter counter ZBII (Beckman Coulter) and by hemocytometer.

1. A pharmaceutical composition comprising an anti-BCLP antibody specific for cells of a cancer, wherein the antibody specifically binds to a polypeptide having an amino acid sequence of SEQ ID. NO: 2, 12, 13, 14, 15, 16, 17, 18 or 22.

2. The pharmaceutical composition of claim 1, wherein the cancer is selected from a group consisting of colon, breast, lung, ovary, prostate, pancreas and skin cancers.

3. The pharmaceutical composition of claim 1, wherein the antibody is a monoclonal anti-BCLP antibody or fragment thereof.

4. The pharmaceutical composition of claim 1, wherein the antibody is labeled with a radioisotope.

5. The pharmaceutical composition of claim 1, wherein the antibody is labeled with a toxin.

6. The pharmaceutical composition of claim 1, wherein the antibody is conjugated to a prodrug activating enzyme.

7. A method of targeting BCLP protein on cells of a cancer, comprising contacting the cells with a composition comprising an antibody that specifically binds to a polypeptide having an amino acid sequence of SEQ ID NO: 2, 12, 13, 14, 15, 16, 17, 18 or 22 or an immunogenic fragment thereof.

8. The method of claim 7, wherein the cancer is selected from a group consisting of colon, breast, lung, ovary, prostate, pancreas and skin cancers.

9. A method of killing or inhibiting the growth of BCLP-expressing cells of a cancer comprising contacting the cells with a composition comprising an antibody that specifically binds to a polypeptide having an amino acid sequence of SEQ ID NO: 2, 12, 13, 14, 15, 16, 17, 18 or 22 or an immunogenic fragment thereof.

10. The method of claim 9, wherein the cancer is selected from a group consisting of colon, breast, lung, ovary, prostate, pancreas and skin cancers.

11. A method of killing or inhibiting the growth of BCLP-expressing cells of a cancer, comprising the step of contacting the cells with a composition comprising a conjugate comprising an antibody linked to a prodrug activating enzyme, and a prodrug convertible under the influence of the conjugate into a cytotoxic drug.

12. The method of claim 11, wherein the cancer is selected from a group consisting of colon, breast, lung, ovary, prostate, pancreas and skin cancers.

13. A method of killing or inhibiting the growth of BCLP-expressing cells of a cancer, comprising the step of contacting the cells with a composition comprising an anti-BCLP antigen.

14. The method of claim 13, wherein the cancer is selected from a group consisting of colon, breast, lung, ovary, prostate, pancreas and skin cancers.

15. A method of killing or inhibiting the growth of BCLP-expressing cells of a cancer, comprising the step of contacting the cells with a composition comprising a nucleic acid of SEQ ID NO: 1, 3, 4, 5, 6, 7, 8, 9, 10, 11, or 19 encoding BCLP, or immunogenic fragment thereof, within a recombinant vector.

16. The method of claim 15, wherein the cancer is selected from a group consisting of colon, breast, lung, ovary, pancreas, prostate and skin cancers.

17. A method of killing or inhibiting the growth of BCLP-expressing cellsof a cancer, comprising the step of administering a composition to the cells, wherein the composition comprises an antigen-presenting cell comprising a nucleic acid of SEQ ID NO: 1, 3, 4, 5, 6, 7, 8, 9, 10, 11, or 19 encoding BCLP, or immunogenic fragment thereof, within a recombinant vector.

18. The method of claim 16, wherein the cancer is selected from a group consisting of colon, breast, lung, ovary, pancreas, prostate and skin cancers.

19. A method of killing or inhibiting the growth of BCLP-expressing cells of a cancer, comprising the step of administering a composition to the cells wherein the composition comprises a small molecule that specifically binds to a polypeptide having an amino acid sequence of SEQ ID NO: 2, 12, 13, 14, 15, 16, 17, 18, or 22 or an immunogenic fragment thereof.

20. The method of claim 19, wherein the cancer is selected from a group consisting of colon, breast, lung, ovary, pancreas, prostate and skin cancers.

21. A method of killing or inhibiting the growth of BCLP-expressing cells of a cancer comprising the step of administering a composition to the cells wherein the composition comprises a polypeptide that specifically binds to a polypeptide having an amino acid sequence of SEQ ID NO: 2, 12, 13, 14, 15, 16, 17, 18, or 22 or an immunogenic fragment thereof.

22. The method of claim 21, wherein the cancer is selected from a group consisting of colon, breast, lung, ovary, pancreas, prostate and skin cancers.

23. The method according to any one of claims 7, 9, 11, 13, 15, 17, 19, or 21, wherein the cells are contacted with as second therapeutic agent.

24. The method according to any one of claims claims 7, 9, 11, 13, 15, 17, 19, or 21, wherein the anti-BCLP antibody composition is administered in an amount effective to achieve a dosage range from about 0.1 to about 10 mg/kg body weight.

25. The method according to any one of claims 7, 9, 11, 13, 15, 17, 19, or 21, wherein the composition is administered in a sterile preparation together with a pharmaceutically acceptable carrier

26. A method of diagnosing a cancer of the colon, breast, lung, ovary, pancreas, prostate or skin, comprising the steps of:
(a) detecting or measuring the expression of BCLP by cells of the cancer; and
(b) comparing the expression to a standard indicative of the cancer.

27. A method of diagnosing a cancer of the colon, breast, lung, ovary, pancreas, prostate or skin, comprising the steps of:
(a) detecting or measuring the expression of BCLP by cells of the cancer; and
(b) comparing the expression to normal tissue.

28. The method of claim 26 or 27, wherein the step of detecting or measuring comprises BCLP mRNA.

29. The method of claim 26 or 27, wherein the step of detecting or measuring comprises BCLP protein.
